(19) United States
(12) Patent Application Publication
(10) Pub. No.: US 2018/0147039 A1
(43) Pub. Date: May 31, 2018

(54) SYSTEM AND METHOD FOR MEASURING AND SIMULATING MANDIBULAR MOVEMENT

(71) Applicants: Gary L. Carlson, Eden Prairie, MN (US); Bruce W. Hultgren, Victoria, MN (US); Steven M. Rzepecki, Eden Prairie, MN (US); Michael C. Marshall, Sheboygan, WI (US); Timothy Hillukka, Minnetonka, MN (US)

(72) Inventors: Gary L. Carlson, Eden Prairie, MN (US); Bruce W. Hultgren, Victoria, MN (US); Steven M. Rzepecki, Eden Prairie, MN (US); Michael C. Marshall, Sheboygan, WI (US); Timothy Hillukka, Minnetonka, MN (US)

(21) Appl. No.: 15/578,206

(22) PCT Filed: May 27, 2016
(86) PCT No.: PCT/US2016/034761

§ 371 Date: Nov. 29, 2017

Related U.S. Application Data
(60) Provisional application No. 62/167,924, filed on May 29, 2015.

(51) Int. Cl. A61C 19/045; A61C 19/05

(52) U.S. Cl. CPC A61C 19/045; A61B 5/0073; A61C 19/05

(57) ABSTRACT

The disclosure provides an improved bite information collection apparatus. In some embodiments, the bite information collection apparatus includes an upper face bow coupled to an upper clutch and a lower face bow coupled to a lower clutch. Additionally the bite information collection apparatus can be configured to a system for digitally retrieving and analyzing a patient's bite. The systems and methods described better simulate motion by using data to replicate bite motion on an improved automated articulator, which can be retrieved by digital or mechanical means.

### CROSS-REFERENCE TO RELATED APPLICATIONS

This application is being filed on 27 May 2016, as a PCT International patent application, and claims priority to U.S. Provisional Patent Application No. 62/167,924 filed May 29, 2015, and titled SYSTEM AND METHOD FOR MEASURING AND SIMULATING MANDIBULAR MOVEMENT, the disclosure of which is hereby incorporated by reference in its entirety.

### INTRODUCTION

Dentistry instruments can be used to provide a better means of simulating “mandibular movement,” or the ways in which the lower jaw moves, in order to provide a higher quality of treatment of dental occlusion. The mandibular fossae and condyles vary amongst each individual. This variation includes different anatomic forms in the anatomical structure of the right and left temporomandibular (tm) joint. In some instances, differences between the right and left temporomandibular (tm) joint in the same individual may also be observed. The orientation of the mandibular fossae (i.e. steep or shallow) influences the amount of overbite that is necessary to build into the occlusion plan so the posterior teeth disclude or clear during protrusion. Therefore, each individual's mandibular movement is unique.

Dental articulators are used in dentistry to reproduce recorded positions of the mandible moving in relation to the maxilla. Articulators attempt to simulate the patient's mandibular movement by hand manipulation. Semi-adjustable and fully-adjustable articulators attempt to simulate the patient's mandibular movement side to side and up and down by adjusting representative factors such as the condylar angle, Bennett side-shift, incisal and cuspid guidance and an approximation to the shape of the glenoid fossae and eminentia. It is difficult, however, to replicate the mandibular and maxilla orientation using the classical parameters available to dental professionals. These standard parameters provide an approximation of mandibular/maxillary orientation and movement based on average movement across groups of patients for part of or all of the mandibular motion.

Traditional static descriptions of jaw joint/dentition relationships (3D geometry) provided by conventional articulators are inaccurate when attempting to determine or predict basic elements of jaw motion. The present subject matter views the orofacial skeleton/dentition as a mechanism. This information is foundational to digitally performing all reconstruction procedures in dentistry. This mechanism has a number of characteristics, such as, for example: mechanism geometry or morphology and kinematics. The present subject matter captures the mechanism geometry or morphology relationship with a Mandibular Movement Recorder (MMR) device and fixed clutch/MMR fiducial registration system. Kinematics is the study of relative motion; the degree to which an object rotates and/or translates between any two points in time is defined by an instantaneous center of rotation (e.g., screw axis). The present subject matter captures, for example, the instantaneous center of rotation in real time with the MMR device/software system. For example, a kinematic algorithm records the instantaneous curvilinear motion of the mandible.

What is needed is a system and method for more accurately measuring and simulating mandibular movement.

### SUMMARY

The system and methods described herein provides an improved bite information collection apparatus. In some embodiments, the bite information collection apparatus includes an upper face bow coupled to an upper clutch and a lower face bow coupled to a lower clutch. Additionally, the bite information collection apparatus can be configured to a system for digitally retrieving and analyzing a patient's bite. The systems and methods described better simulate motion by using data to replicate bite motion on an improved automated articulator, which can be retrieved by digital or mechanical means.

### BRIEF DESCRIPTION OF THE DRAWINGS

FIG. 1A illustrates one embodiment of a bite information collection apparatus.

FIG. 1B illustrates one alternative embodiment of a bite information collection apparatus.

FIG. 1C is an illustration of one alternative embodiment of the lower bow.

FIG. 1D is an illustration of one alternative embodiment of the upper bow.

FIG. 1E is an illustration of a more detailed view of the magnetized component, clutches and the upper and lower bows.

FIG. 1F is an illustration of an exploded view of one embodiment of the magnetized component.

FIG. 1G is an illustration of an exploded view of one embodiment of the magnetized component.

FIG. 2A is an illustration of one embodiment of a laser housing.

FIG. 2B is an illustration of one embodiment of a laser housing attached to a side arm.

FIG. 3A is an illustration of one embodiment of the fiducials of the mandibular movement system.

FIG. 3B is an illustration of one embodiment of the fiducials of the mandibular movement system.

FIG. 4A is an illustration of a side elevation view of the relationship between clutches and a condylar axis.

FIG. 4B is an illustration of the top perspective view of the relationship between clutches and a condylar axis.

FIG. 5A is an illustration of the side elevation view of the relationship between clutches in a proximal position.

FIG. 5B is an illustration of the side elevation view of the relationship between clutches in a distal position.

FIG. 5C is an illustration of the anterior-posterior and left-right plane trace.

FIG. 6 is an illustration of a bite information collection system.

FIG. 7A is an illustration of a top plan view of right and left gripping elements fit against the facial surfaces of the cuspids, bicuspids and first molars.

FIG. 7B is an illustration of a side elevation view of right and left gripping elements fit against the facial surfaces of the cuspids, bicuspids and first molars.

FIG. 7C is an illustration of a front elevation view of a clutch comprising a bearing point and bearing surface.

FIG. 7D is an illustration of a side elevation view of a clutch comprising a bearing point and bearing surface.

FIG. 7E is an illustration of a maxillary clutch with indexing grooves.

FIG. 7F is an illustration of a maxillary clutch with indexing grooves.

FIG. 7G is an illustration of a mandibular clutch with indexing grooves.

FIG. 7H is an illustration of a mandibular clutch with indexing grooves.

FIG. 7I is an illustration of a clutch comprising a plurality of adhesive bonding screws.

FIG. 7J is an illustration of a top view of another embodiment of clutch.

FIG. 7K is an illustration of a side view of another embodiment of clutch.

FIG. 8A is an illustration of a process of using a bite information collection system.

FIG. 8B is an illustration of a process of using a bite information collection system.

FIG. 9 is an illustration of the side elevation view of one embodiment of an upper face bow.

FIG. 10 is an illustration of another embodiment of an upper face bow within a digital bite information collection apparatus.

FIG. 11A is an illustration of a top plan view of a clutch connection mechanism than includes a nasion rest.

FIG. 11B is an illustration of a side elevation view of a clutch connection mechanism.

FIG. 11C is an illustration of a front elevation view of a clutch connection mechanism.

FIG. 11D is an illustration of a side elevation view of a clutch connection mechanism.

FIG. 12 is a top perspective view of a bitefork assembly with segmented assembly arms.

FIG. 13 is an illustration of a side perspective view of a fossa simulator.

FIG. 14 is an illustration of a top perspective view of the lower section of a simulator.

FIG. 15 is an illustration of a side perspective view of a fossa simulator.

FIG. 16A is an illustration of a side perspective view of a bite-tray style clutch.

FIG. 16B is an illustration of a side perspective view of an alternative embodiment of a bite-tray style clutch.

FIG. 16C is an illustration of a bottom plan view of a bow attached to a clutch.

FIG. 16D is an illustration of a side elevation view of a bow attached to a clutch.

FIG. 17 illustrates a block diagram of an example circuit which can be used in conjunction with a bite information collection system.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

Dental care providers often create a three dimensional (“3D”) physical model or cast of one or more areas of a patient's oral cavity. With a 3D physical model, a care provider can interact with the model to quickly view multiple angles of the model and to visualize adjustments made to the model. For example, care providers may create a model of an area of a patient's oral cavity where one or more teeth are missing or damaged, so that suitable replacement teeth may be made in the lab using the model as a guide.

Proper fitting the lab-made teeth would benefit from an understanding of how the teeth are used by the patient. In other words, somehow linking the model to the mechanics of the patient's bite is desirable. As noted above, in the past, care providers have used dental articulators in conjunction with the models to replicate movement of the patient's mandible about a hinge axis. This is a crude replication of the bite and often fails to capture the intricacies of the bite motion, including habitual adaptation of the bite to malformations of the teeth. Habitual adaptation of the bite often occurs via muscle training. In addition to failing to capture the complex curvature of the bite, the known methods are imprecise and inaccurate, as the measurement device is often mounted to soft tissue and data is recorded via a pen, and other factors introducing error during the procedure. In addition error is introduced by incorrect assumptions during model creation.

Systems and methods for capturing dental articulation are described in U.S. Pat. No. 8,556,626, issued Oct. 15, 2013 to Evenson and in U.S. Pat. No. 8,834,157, issued Sep. 16, 2014 to Evenson et al., the discussions of which are incorporated herein by reference. The systems and methods described allow care providers to understand articulation, create models of that articulation, find improved articulation if possible, and provide treatments based on the models that either allows existing articulation or encourages improved articulation.

The aforementioned systems and methods capture bite motion by fixing measuring tools to both the top and bottom teeth. This is preferred over systems that fix to only one dental arch, as it reduces error. Systems that fix to only one dental arch inevitably fix the other portion of a bite monitoring system to soft tissue, which can result in error via, for example, tissue movement. The approach described herein replicates the function of the condylar axis, which is the axis of rotation of the mandible around the mandibular condyle. The approach reduces reliance on soft tissue and other error prone data in establishing the geometric relationship of both rows of teeth to one another throughout a range of mandibular articulations.

The systems and methods described also better simulate motion by using digital data to replicate bite motion on an improved automated articulator, which, in some embodiments, includes a Stewart platform. By recording bite motion in a digital format, and then simulating the bite motion using the digital data, at least one opportunity for error is eliminated. Data from the monitor is transcribed to the articulator digitally, rather than being replicated by hand adjustments to the articulator.

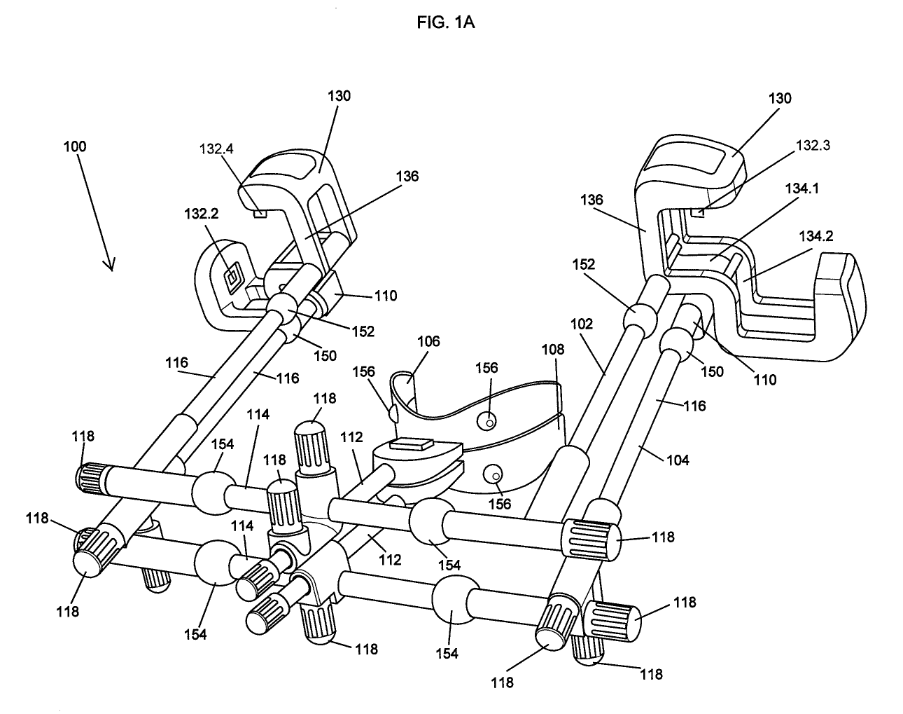

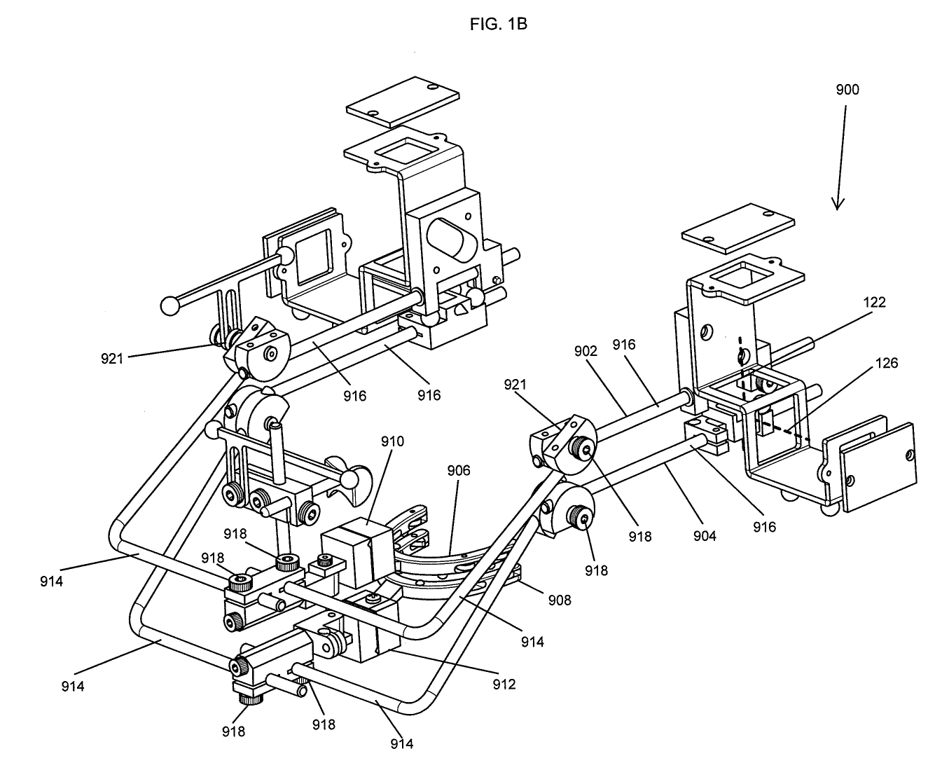

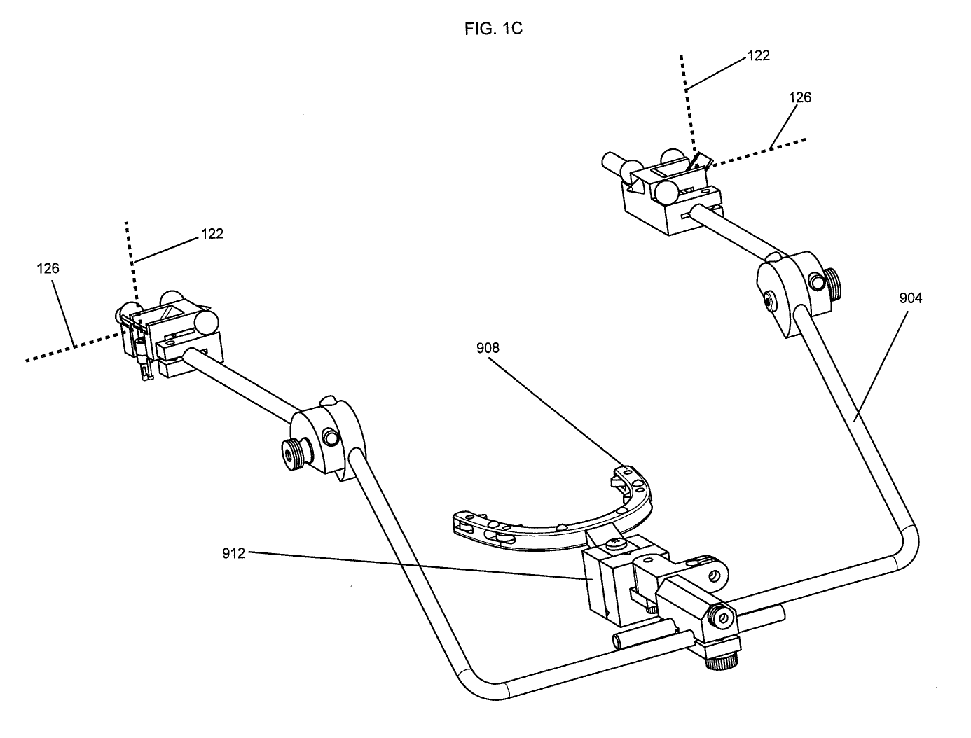

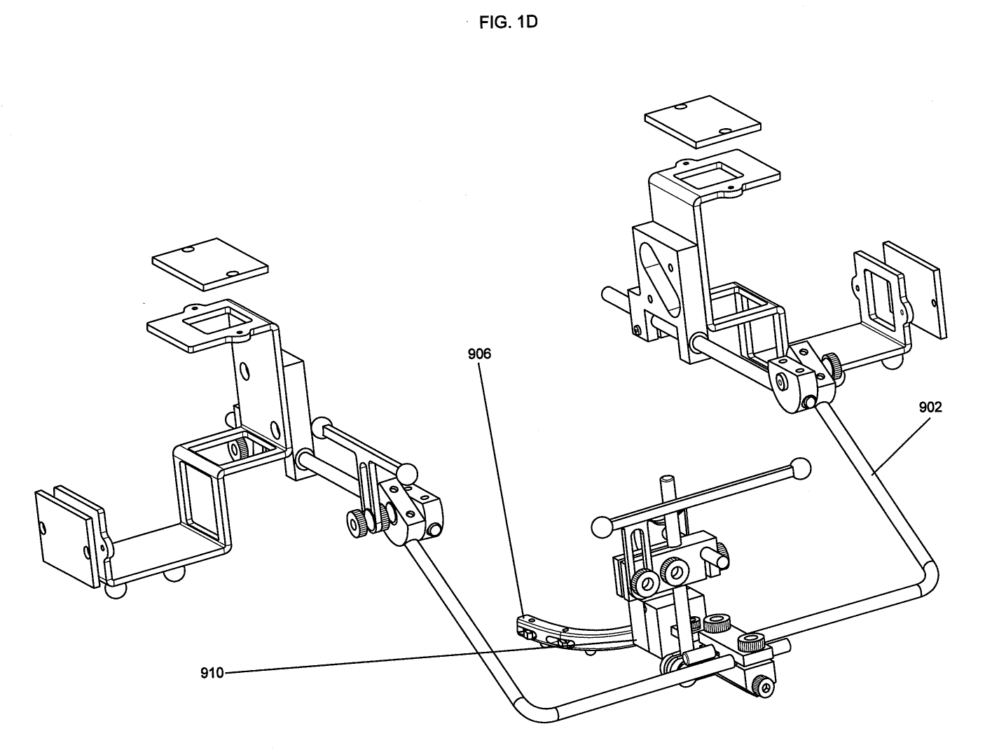

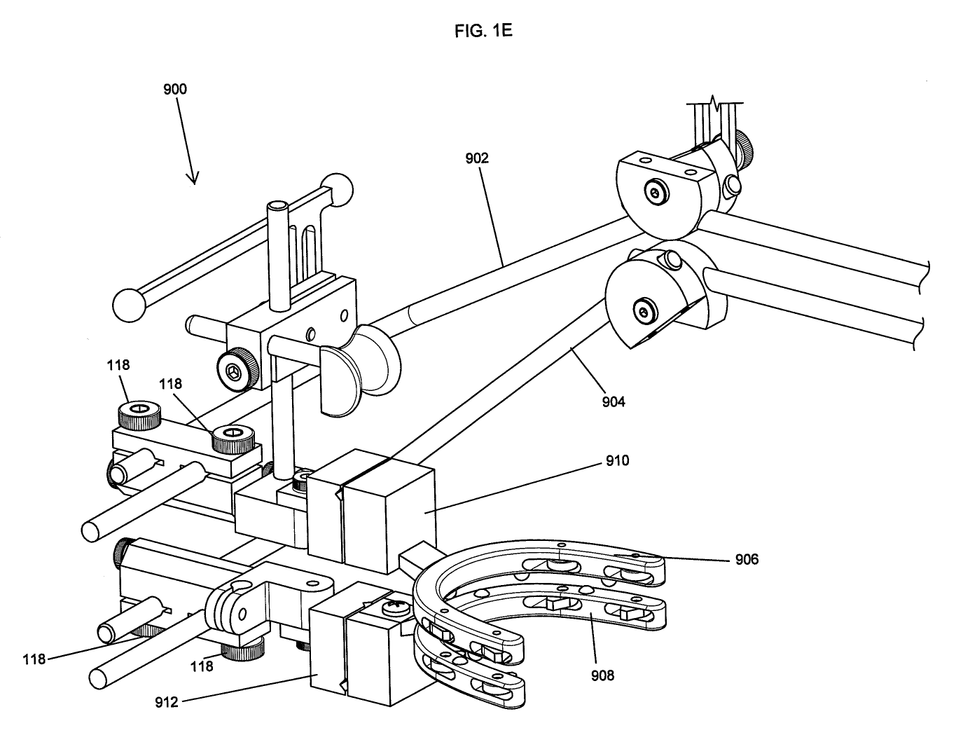

FIG. 1A is an illustration of a bite information collection apparatus 100, according to some embodiments. In some embodiments, apparatus 100 includes an upper face bow 102 coupled to an upper clutch 106 and a lower face bow 104 coupled to a lower clutch 108. In one example embodiment, the upper clutch is coupled to the upper teeth and, optionally, to gingiva of a patient, such as by using a quick setting, compliant compound that can both capture the shape of the teeth and which can be released from the teeth. The lower clutch is coupled to the bottom teeth of a patient, and, in some embodiments, includes a quick setting, compliant compound disposed therein, in a manner similar to upper clutch 106.

In some embodiments, the care provider first mounts lower clutch 108 and upper clutch 106 to the patient's teeth and then attaches upper face bow 102 and lower face bow 104 to their respective clutches. In some embodiments, each clutch is connected to its face bow via clutch rods 112. Still referring to FIG. 1A, face bows 102 and 104 include an anterior rod 114 connected to two side arms 116 and to one of the clutch rods 112. Fasteners 118 permit easy adjustment of apparatus 100.

In some embodiments, a laser is used to determine how the condyle moves within the joint. In some such embodiments, one of the face bows 102, 104 includes a laser housing 110 that projects laser light 122 in a direction parallel to the sagittal plane and projects laser light 126 in a direction approximately orthogonal to the sagittal plane.

In one embodiment, as is shown in FIG. 1A, apparatus 100 includes fiducial markings for indicating the mechanical geometry of various components. In the example embodiment shown in FIG. 1A, fiducials 150 are used to indicate spatial relation of the lower teeth to the hinge axis. Fiducials 152 are used to indicate spatial relation of the upper teeth to the hinge axis. Fiducials 154 are used to indicate spatial relation of a centerline through clutches 106 and 108 and their corresponding side arms 116. Finally, fiducials 154 and 156 are used to indicate spatial relation from anterior rods 114 to their respective clutches 106 and 108.

In at least one example embodiment, fiducials 150, 152, 154 and 156 capture the position via a camera and the spatial relation of the lower teeth to the hinge axis, from the upper teeth to the hinge axis, from a centerline through clutches 106 and 108 and their corresponding side arms 116 and from anterior rods 114 to their respective clutches 106 and 108 is determined. In a related embodiment, fiducials 150, 152, 154 and 156 are scanned via a Leap Motion scanner to determine each of the above measurements. Such an approach is one mechanism for placing upper face bow 102 and lower face bow 104, and their respective clutches 106 and 108 in three-dimensional space. The Leap Motion device is available from Leap Motion, Inc., of San Francisco, Calif. In another related embodiment, fiducials 150, 152, 154 and 156 are designed to be scannable from many different angles. For example, some or all of fiducials 150, 152, 154 and 156 are approximately spherical in shape and have indicia for enhancing capture of the location of the fiducial. Other methods for capturing the location of fiducials such as fiducials 150, 152, 154 and 156 are contemplated as well. For instance, in some embodiments, laser scanning is used to build a three-dimensional model of apparatus 100. In some embodiments, optical scanners are used to capture fiducial locations. In other embodiments, a computerized tomography such as flash CT captures the location of the fiducials.

In one example embodiment, touch probes such as those manufactured by Renishaw PLC of Hoffman Estates, Illinois are used to capture the location of fiducials such as fiducials 150, 152, 154 and 156. A touch on each of fiducials 150, 152, 154 and 156 as shown in FIG. 1A would quickly and accurately locate the center of each fiducial.

The face bow includes laser housing 110 and further comprises camera housing 130. Either laser housing 110 or camera housing 130 may be used without the other. In the embodiment shown in FIG. 1A, camera housing 130 includes cameras 132 and translucent screens 134. In some such embodiments, translucent screens 134.1 and 134.2 are arranged between lasers 120 and 124 and cameras 132.1 (not shown) and 132.2, 132.3 and 132.4 so that light 122 and light 126 fall on screens 134.1 and 134.2, respectively, to be captured by cameras 132.1 (not shown) and 132.2, 132.3 and 132.4, respectively. In some embodiments, translucent screens 134 include a grid pattern used to assign a location to the spot where light 122 and 126 pass through screens 134.1 and 134.2, respectively.

FIG. 1B is an illustration of a bite information collection apparatus 900, according to some embodiments. Although not every component is discussed it should be understood that the bite information collection apparatus 900 includes similar components as the bite information collection apparatus 100 discussed in reference to FIG. 1A. For example, the bite information collection apparatus 900 includes an upper face bow 902 coupled to an upper clutch 906 and a lower face bow 904 coupled to a lower clutch 908. In one embodiment, the face bows 902, 904 are each coupled to the clutches 906, 908, respectively, by a magnetized component 910, 912. Further, bite information collection apparatus 900 comprises fasteners 918 permit easy adjustment of apparatus 900. In at least this example embodiment fiducials can be used to indicate spatial relation of the upper teeth to the hinge axis. The spatial relation of a centerline through clutches 906 and 908 and their corresponding side arms 916. The spatial relation from anterior rods 914 to their respective clutches 906 and 908 may also be determined. It should be appreciated that in at least the embodiment shown in FIG. 1B, the upper face bow 902 and lower face bow 904 have at least one rotational hinge 921 connecting the side arms 916 and anterior rods 914. Further, where fiducials are shown, for example along the anterior rod in FIG. 1A, fasteners 918 are used to tighten face bow in a desired position once the bite information collection system is properly aligned. In some embodiments, the proper alignment of the bite information collection system comprises adjusting the rotational hinge 921 so that the side arms 916 are oriented approximately horizontally. In this manner, the laser housing 110 and the camera housing 130 will be positioned in approximately the same orientation thus allowing the laser beams 122 and 126 emitted by the laser housing 110 to contact the translucent screens (e.g., the translucent screens 134.1, 134.2 of the camera housing 130.

FIG. 1C is an illustration of an example of the lower face bow 904, including the clutch 908 coupled by the magnetized component 912. A touch probe inserted into cavity 157 that comes in contact with the bottom of cavity 157 would determine, with great accuracy, the location of the center of fiducials 156. FIG. 1D is an illustration of an example of the upper face bow 902, including the clutch 906 coupled by the magnetized component 910. FIG. 1E is an illustration of a more detailed view of the magnetized component 910, 912, clutches 906, 908, and the upper and lower face bows 902, 904. Referring specifically to FIG. 1E, where fasteners 118 permit easy adjustment of apparatus 100. In at least this example embodiment, the fasteners 118 adjust anterior rods 114 that are in different vertical planes. It should be appreciated that the ability to determine the mandibular and maxillary orientations can be calibrated via mechanical or digital means in embodiments where anterior rods 114 are in the same vertical planes or different vertical planes.

FIGS. 1F and 1G illustrate an exploded view of the magnetized component 910. Although magnetized component 910 is illustrated, it is to be understood magnetized 912 is similar in form and function. As shown in FIG. 1F, the magnetized component 910 includes a first metallic portion 920 and a second metallic portion 926. In an example, the first portion 920 includes at least one mating feature 922, such as a protrusion. In an example, the magnetized component 910 includes a magnet 924. The second metallic portion 926 includes a coupling feature 930 for coupling with a clutch (906, 908—not shown) and at least one second mating feature 928. Referring to FIG. 1G, the second mating feature 928, in an example, includes a v-shaped groove, a recess to receive a corresponding protrusion, or the like. In one embodiment, the first and second mating features 922 and 928 are configured to align the first and second metallic portions 920, 926 in a horizontal and a vertical direction. In one embodiment, the magnet 924 is at least partially received in a magnet orifice 932 of the second metallic portion 926. In other related embodiments magnet 924 is at least partially received in a magnet orifice 932 of the first metallic portion 920.

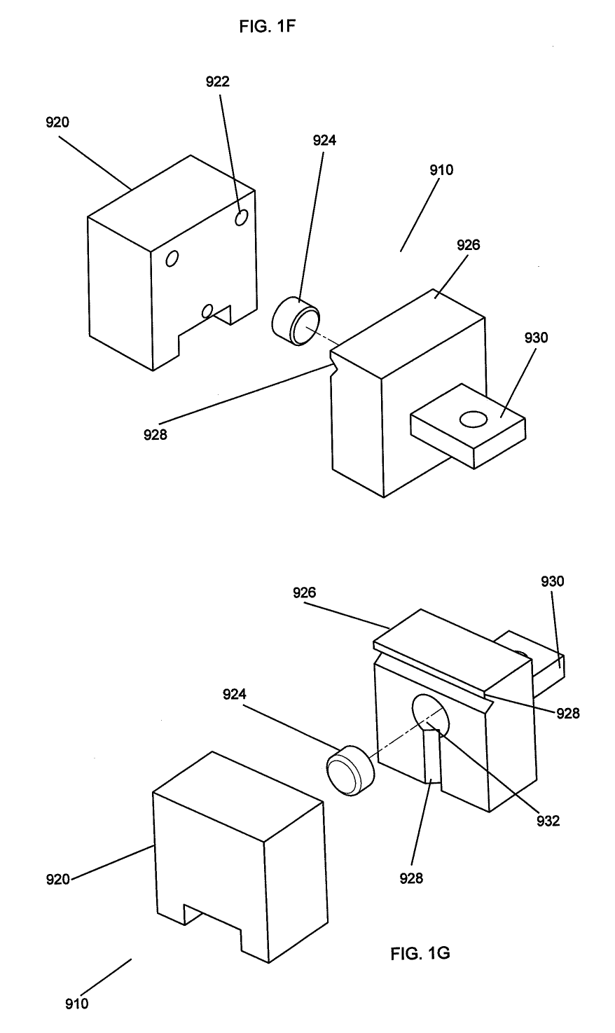

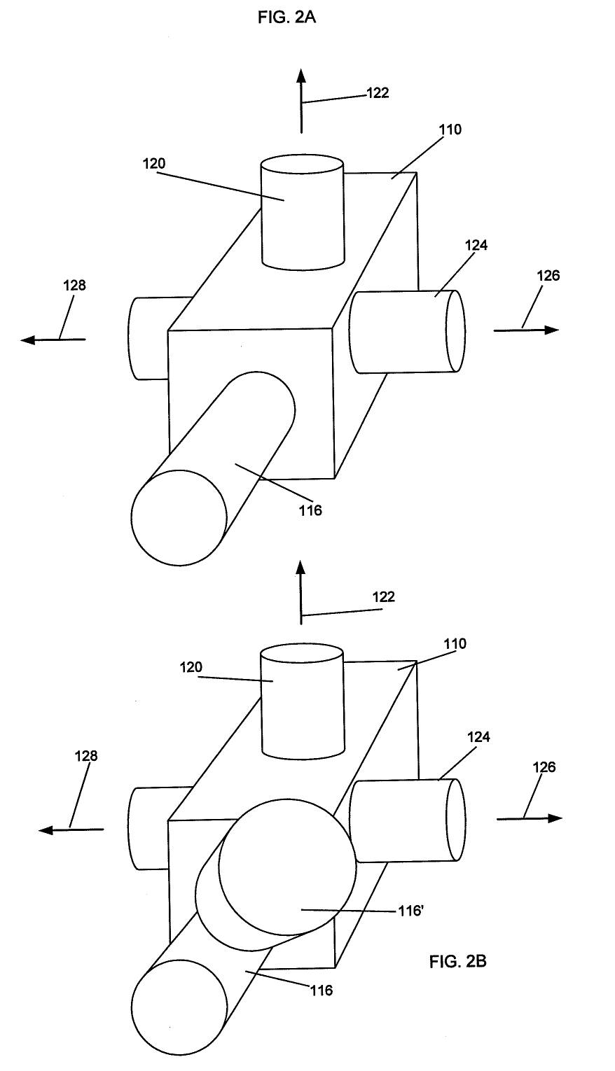

As shown in FIG. 2A, laser housing 110 includes a first laser 120 that projects light 122 upward parallel to the sagittal plane and a second laser 124 that projects light 126 out in a direction orthogonal to the sagittal plane and away from the patient's head. In one such embodiment, laser 124 is collinear. That is, laser 124 sends light in two directions: outward as in light 126 in FIG. 2A and toward the patient as in light 128 in FIG. 2A. In some such embodiments, light 128 is used to trace movement of the condyle on the patient's soft tissue and can be used to guide the doctor in placing a mark on the location of the axis of the mandibular condyles. In one embodiment, such as is shown in FIG. 2B, laser housing 110 is mounted to side arm 116 in such a way that laser housing 110 can be placed at an angle from side arm 116 (such as shown in position 116′). In one example embodiment, laser housing 110 is mounted to side arm 116 so that laser housing 110 pivots around side arm 116. Such an approach is useful when the face bow associated with the particular side arm 116 is at an angle outside the expected range of angles. It allows the service provider to angle the laser housing to place it more in line with screens 134.

In one embodiment, the doctor adjusts lower face bow 104 so that laser 120 and laser 124 are in the vicinity of the mandibular condyle. The care provider then adjusts upper face bow 102 so camera housing 130 is placed proximate to lasers 120 and 124. In some embodiments, camera housing 130 includes an anterior-posterior registration screen 134.1 and a horizontal registration screen 134.2, and corresponding cameras 132. In such an embodiment, light 126 from laser 124 falls on anterior-posterior registration screen 134.2 and light 122 from laser 120 falls on a horizontal registration screen 134.1. The light falling on anterior-posterior registration screen 134.2 and horizontal registration screen 134.1 is then captured by an anterior-posterior cameras 132.1 (not shown) and 132.2 and horizontal cameras 132.3 and 132.4 placed in line with light 126 and 122, respectively. These adjustments can optionally be performed by hand (using, e.g., hand adjustable fasteners 118 such as shown in FIG. 1A) or via a motor. This allows the tool to adjust to fit persons of different sizes and in different stages of development. Other mechanisms can be used as well to adjust the length of side arms 116 to place light 128 in the vicinity of the condylar axis.

Although movement of the mandibular condyle is tracked using the first and second lasers 120 and 124 and cameras 134 above, the present subject matter is not so limited. Other optical sensors can be used to track condyle motion through movement of apparatus 100. The sensor fields can include, for example, position sensitive diodes (“PSDs”), but the present subject matter is not so limited.

Other position detecting mechanisms are available as well. In one example embodiment, lasers 120 and 124 are replaced with a painted dot that can be imaged by cameras 132 through screens 134.1 and 134.2. In other example embodiments, lasers 120 and 124 are replaced with collimated light emitting diodes (LEDs) the light from which can be imaged by cameras 132 through screens 134.1 and 134.2. In other example embodiments, lasers 120 and 124 are replaced with a symbol, such as a bar code or a striation that can be imaged by cameras 132 through screens 134.1 and 134.2. In other example embodiments, lasers 120 and 124 are replaced with a fiducial whose position and/or orientation can be imaged by cameras 132 through screens 134.1 and 134.2. In other exemplary embodiments, lasers 120 and 124 are replaced with a single laser which is used with a beam splitter to provide light 122 and 126. In at least this example embodiment, a laser splitter (not shown) is used to direct laser light from one laser to both registration screens 134, but the present subject matter is not so limited.

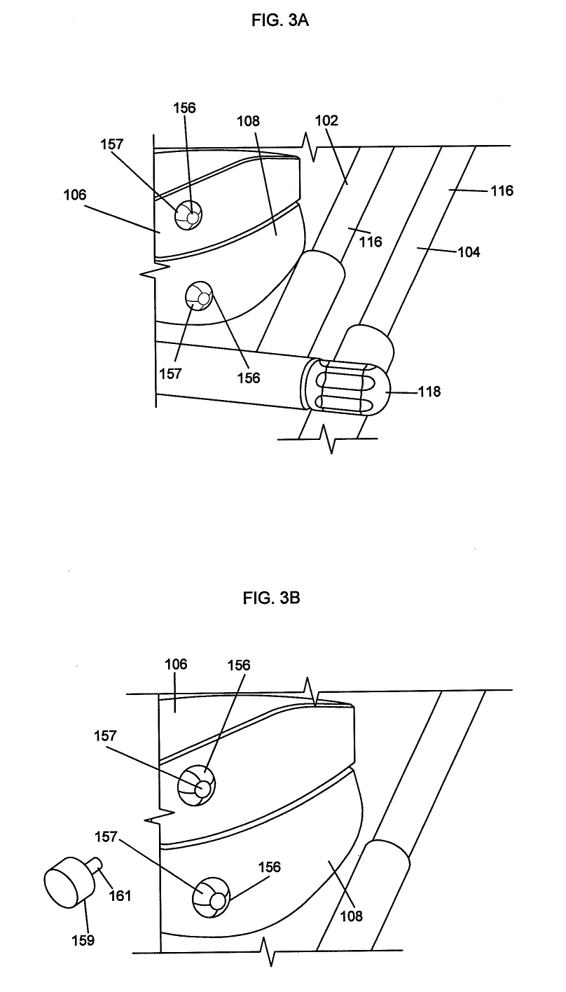

In some example embodiments, it is useful to determine the location and orientation of any touch probe used on fiducials 150, 152, 154 and 156. In such embodiments, cavity 157 and touch probe 159 are keyed so that the orientation of probe 159 is known from the fact that probe tip 161 can enter cavity 157. An example of such an embodiment is shown in FIG. 3B.

A “mandibular movement” might be opening the mouth, moving the mandible in an anterior direction or moving the mandible in a right or left excursive movement. Referring specifically to FIG. 3A, fiducials 156 are spherical in shape and include a cavity 157 that extends into the spherically shaped fiducial. As described above, touch probes such as those manufactured by Renishaw PLC of Hoffman Estates, Illinois may be used to capture the location of fiducials such as fiducials 150, 152, 154 and 156. In the embodiment shown in FIG. 3B, probe tip 161 is a touch probe tip similar to those provided by Renishaw PLC, but designed with a touch probe end having a three-dimensional male/female lock key design that fits into a matching three-dimensional indentation 157 in fiducial 156. In some embodiments, the keyed touch probe is magnetized and articulating on a cable for ease of use. In some such embodiments, the magnetized tip locks the probe into cavity 157 to ensure an accurate measurement. In other embodiments, other retention mechanisms are used to lock the probe into cavity 157. In one embodiment, lower face bow 104 includes a hinge (not shown) that can be used to line up laser 124 with screen 134.2 in situations when lower face bow 104 is at such an angle that light 126 from laser 124 doesn't fall on screen 134.2.

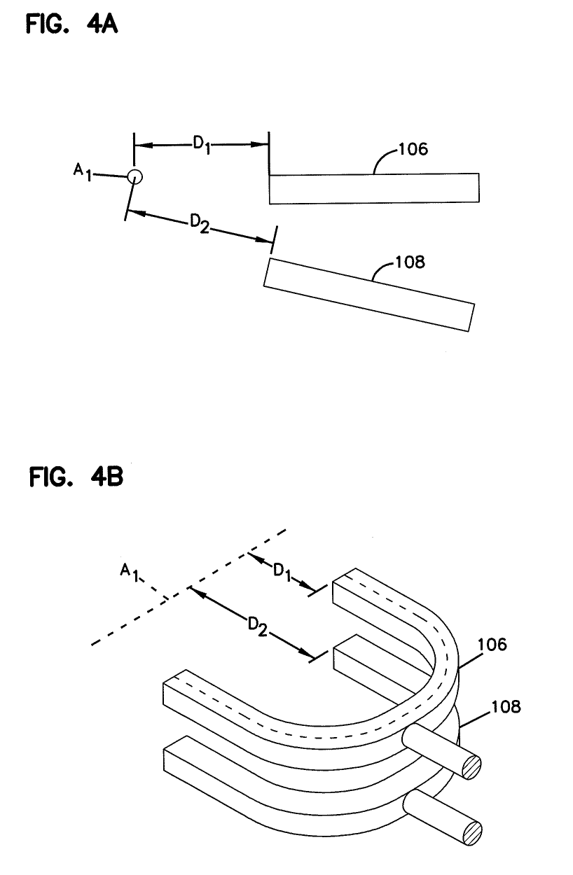

During approximately 10 to 25 mm of opening or closing of the mouth from and to contact of the teeth, the mandible rotates around an axis of rotation A1, (i.e. FIGS. 4A and 4B), which is located in the mandibular condyle. This is often referred to as a “hinge movement,” and therefore, this axis is called the “hinge axis.” When the mandibular condyles are in their anterior-superior, “seated” position in the mandibular fossae, this axis is called the “terminal hinge axis.” This is understood to be the physiologic position from which “mandibular movements” or “excursions” start. Referring to FIG. 4A and 4B which illustrate the relationship between clutches 106 and 108 and a condylar axis A1, according to some embodiments. The present subject matter accurately and precisely captures D1 and D2 by running the laser, broadcasting laser light against screen 134.2 to form a condylar axis trace on screen 134.2, and digitizing and storing the information captured digitally in a measurement computer for analysis and display.

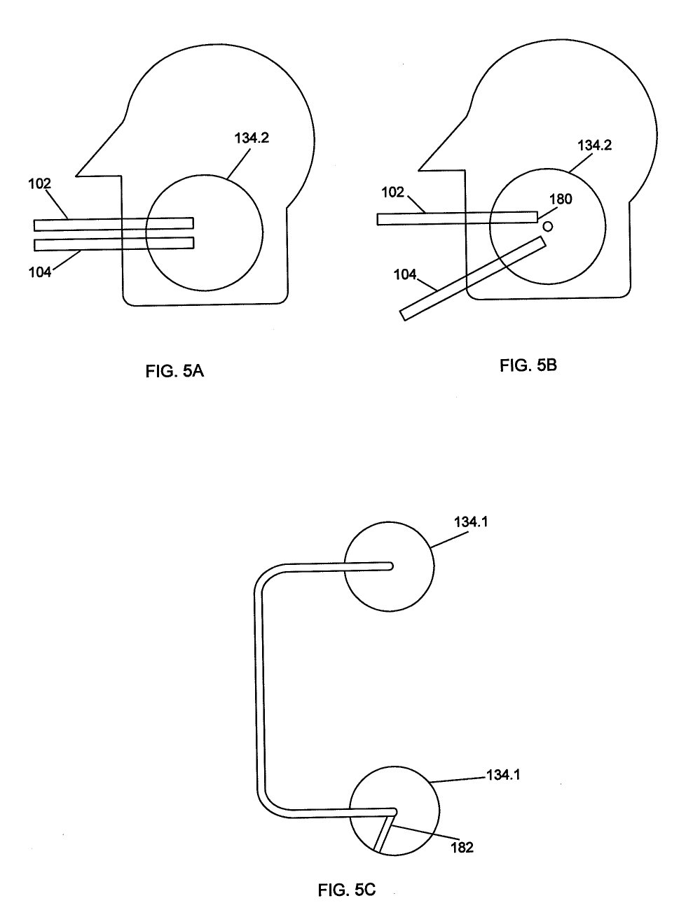

FIGS. 5A and 5B show a pantograph in two separate positions. As patients move their mandibles from the position illustrated in FIG. 5A to the position illustrated in FIG. 5B, lower face bow 104 follows the mandibular movement. A laser (in either face bow 102 or face bow 104) traces the movement as a path 180 of laser light against a sensor plane (e.g., screens 134.2, as shown in FIG. 1A). Camera 132.2 (FIG. 1A) captures this path, in some cases digitally. In some embodiments, precision is to at least 0.0001 of an inch. In the embodiments shown, path 180 is shown in the anterior-posterior/dorsal-ventral plane.

At the same time, laser (in either face bow 102 or face bow 104) traces the movement as a path 182 of laser light against a sensor plane (e.g., screens 134.1) as shown in FIG. 5C. Camera, opposite camera 132.2, captures this path, in some cases digitally. In some embodiments, precision is to at least 0.0001 of an inch. In the embodiment shown, path 180 is shown in the anterior-posterior/left-right plane, as illustrated by trace 182 (FIG. 5C) on screen 134.1 (FIG. 1A). This approach allows for iterative captures, which can statistically improve certainty that the path of interest is captured. In some embodiments, this information is used to study the patient and to provide therapy to the patient. In some embodiments, a measurement computer monitors data and issues an alert to a care provider when a specific degree of statistical certainty as to the arc of the jaw is determined.

In one embodiment, the axis of rotation, or hinge axis, is located, recorded and transferred to an appropriate instrument on which the prosthetic item is to be fabricated.

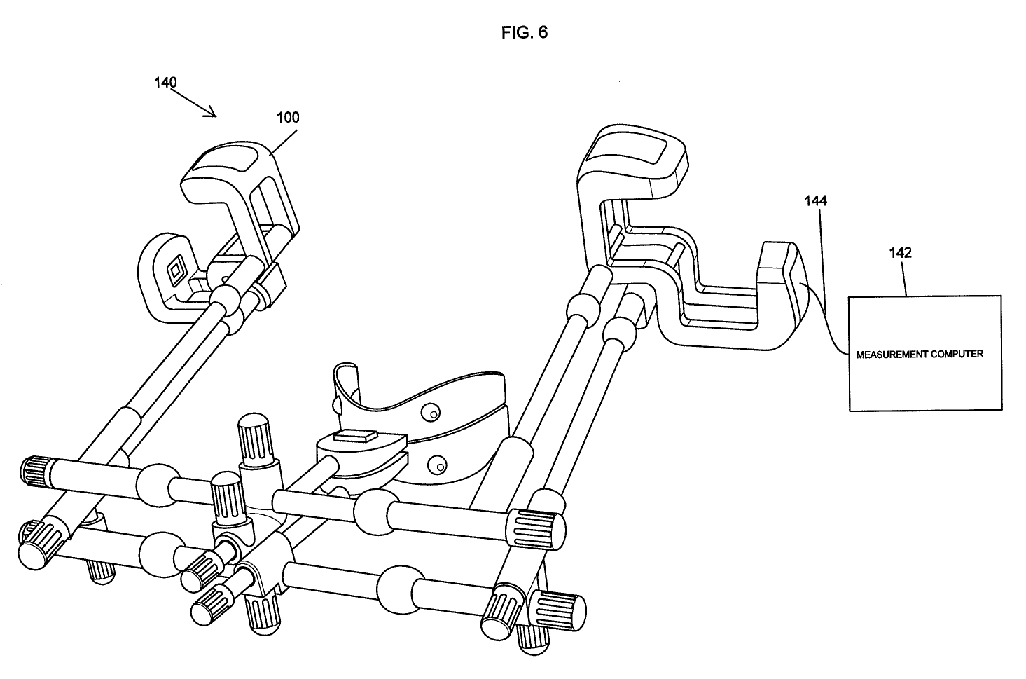

A bite information collection system 140 is shown in FIG. 6. In various embodiments, as is shown in FIG. 6, system 140 includes a measurement computer 142. In some such embodiments, the care provider attaches upper face bow 102 and lower face bow 104 to a patient, checks operation and placement of lasers 120 and 124, and establishes communication between apparatus 100 and measurement computer 142 using communications channel 144. In one such embodiment, cameras 132 communicate with measurement computer 142 in order to convey pictures representing movement of light 126 corresponding to mandibular movement around the condylar axis. In some such embodiments, communications channel 144 includes a wireless transceiver installed in, e.g., camera housing 130. In other embodiments, a wire connects camera 132 to measurement computer 142.

Previous instruments do not locate the axis of rotation accurately and are difficult and time consuming to use. Quite often an instrument is used to determine an “arbitrary” or “estimated” axis of rotation. The problem is that such estimates can vary widely from the actual axis of rotation (from one-half millimeter to two or three millimeters). There will, therefore, always be some degree of error when an “estimated axis” is used. Therefore, in some embodiments, it is critical to accurately locate the physiologic axis of rotation when treating the entire occlusion with a full-mouth reconstruction, orthodontic treatment or any treatment that involves the entire occlusion.

As described herein, one way to address these problems is by helping the care provider to understand the natural path and the habitual path in operation of an articulator. System 140, through tooth scans, can digitize bite motion and store and display that on a measurement computer 142, in conjunction with an actual bite.

Different upper clutches 106 and lower clutches 108 can be used in system 140. In some embodiments, clutches 106 and 108 are facial surface attach clutches attached to one or more teeth. In some such embodiments, the facial surface attach clutches are cemented to the buccal surface of the bicuspids and/or molars to keep them in place.

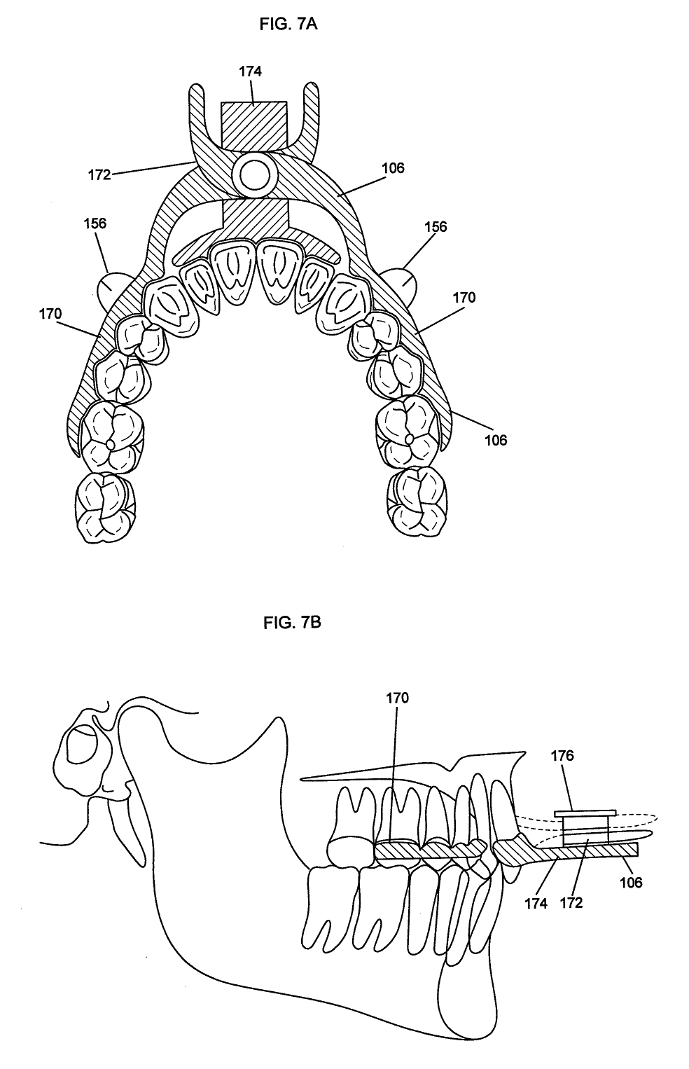

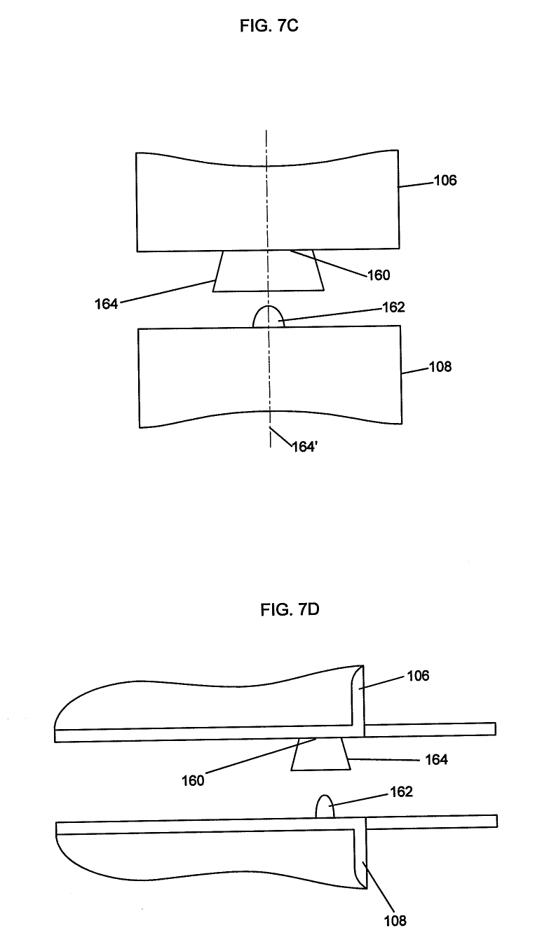

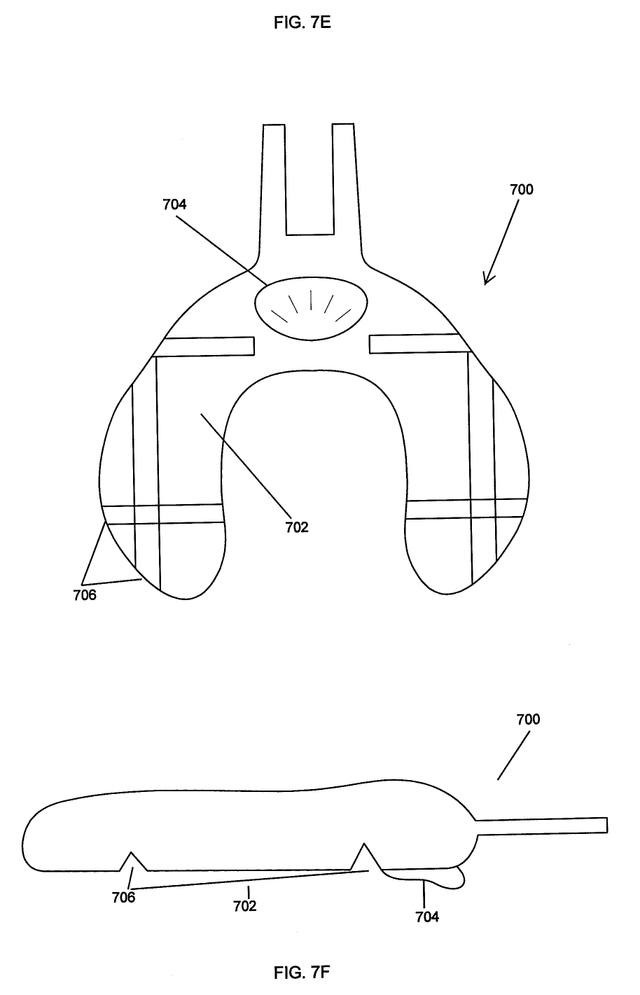

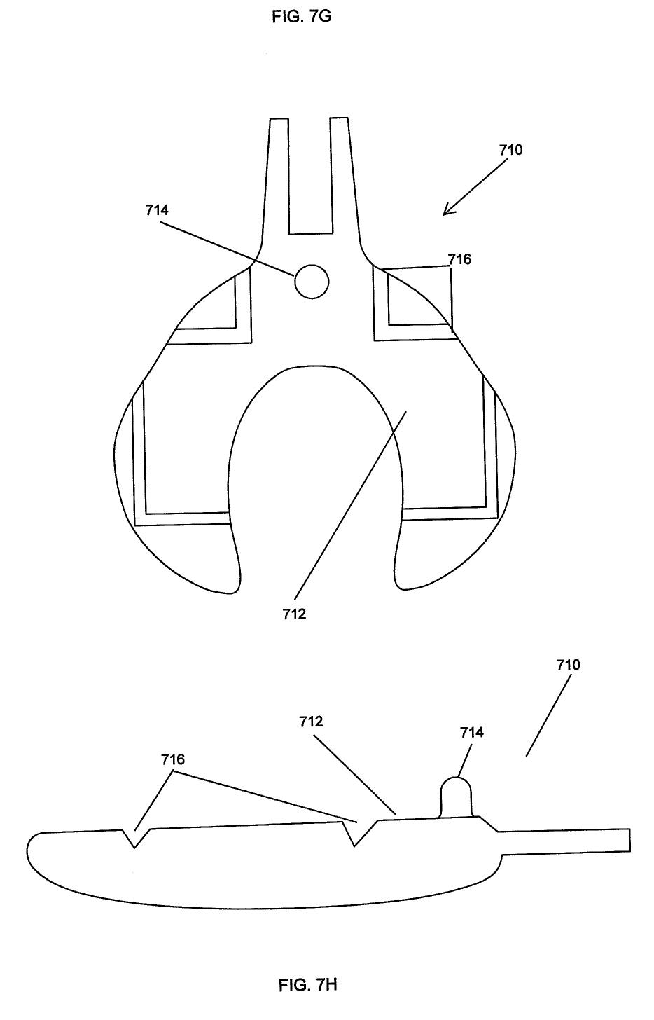

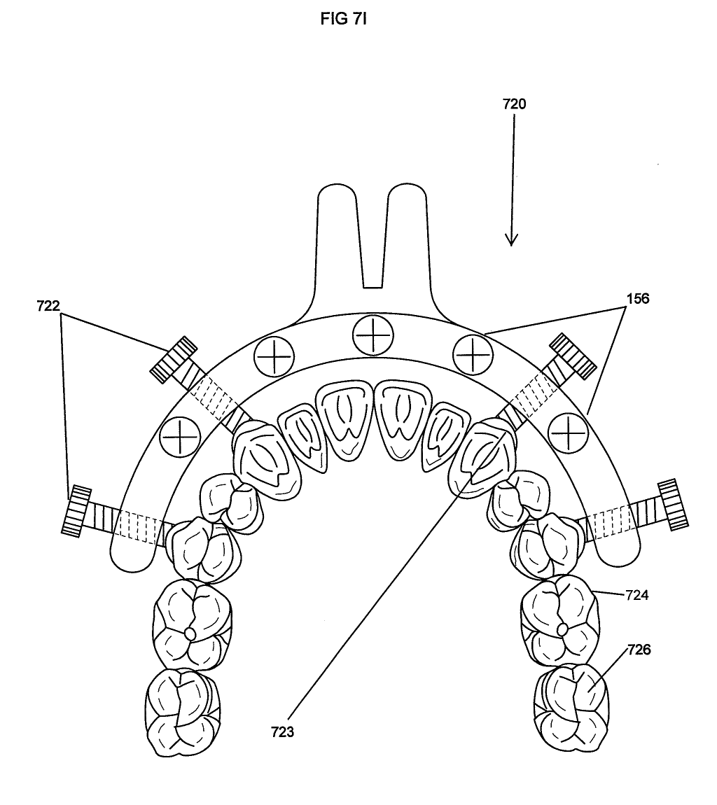

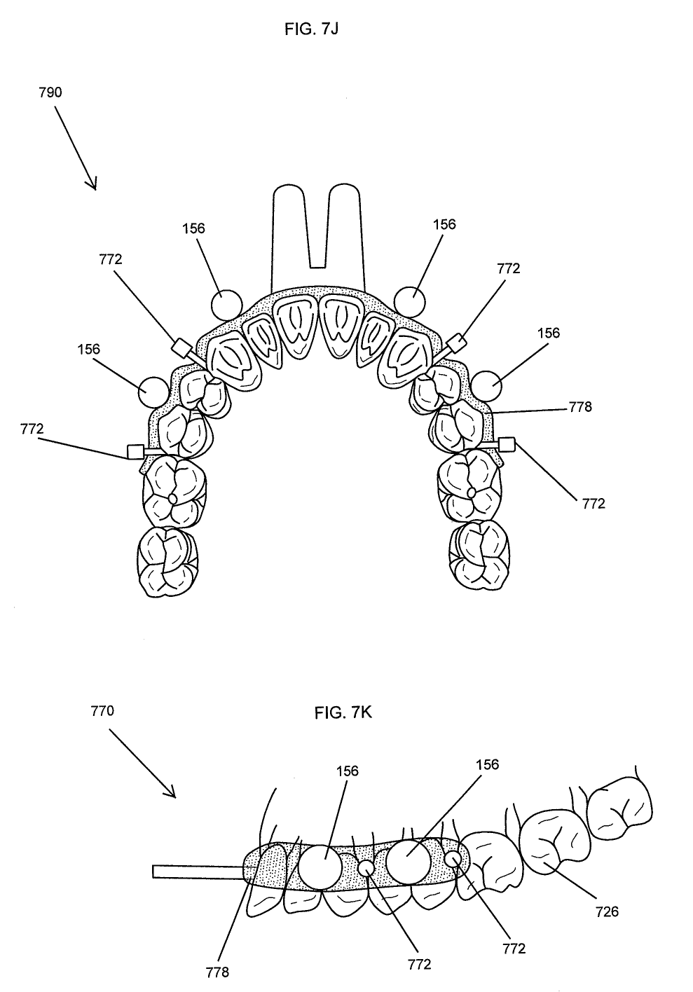

Referring now to FIGS. 7A and 7B where clutches 106 and 108 are held in place via pressure. A clutch 106 that provides for contact of the teeth during a mandibular function recording procedure by realizing retention through tooth surface curvatures and recesses and through pressure adaptation is shown in FIGS. 7A and 7B. In the clutch 106 of FIGS. 7A and 7B, the clutch includes gripping elements 170 with indentations and protrusions that match the patient's teeth. A bottom view of a clutch 106 held in place via pressure is shown in FIG. 7A. In the example embodiment of FIG. 7A, gripping elements 170 operate in a clamping action around a hinge 172. In some such embodiments, clutch 106 includes a locking mechanism for holding the gripping elements 170 in place. In one such embodiment, gripping elements 170 are held in place via a locking mechanism such as is used in surgical clamps. In other such embodiments, a spring mechanism operates on the other side of hinge 172 to apply pressure to gripping elements 170.

In at least one example embodiment, such as is shown in FIGS. 7A and 7B, clutch 106 includes a stabilizing member 174 which is pressed up against the front teeth. In one such embodiment, clutch 106 is designed to fit on both the maxillary and mandibular teeth. In the example embodiment shown in FIGS. 7A and 7B, right and left gripping elements 170 fit against the facial surfaces of the cuspids, bicuspids and first molars. In some embodiments, member 174 and gripping elements 170 are fabricated with a 3D printer and plastic material. In other related embodiments, retention is gained from the fit against the tooth curvature and recesses without any additional retentive material. In some embodiments, this requires a custom fitting to the patient's teeth using, e.g., a digital scanning of the teeth. In other exemplary embodiments, retention is gained from the tooth curvatures and recesses as discussed above and also through the use of a retentive material such as polyvinylsiloxane (PVS) that adheres to a retentive form on the tooth side of the side-arms (a mesh or an lattice with undercuts).

In other example embodiments, as noted above, clutch 106 is a generic clutch that gains retention through a retentive form to the tooth side of elements 170 and the inward pressure of gripping elements 170 against the teeth. In some embodiments, stabilizing member 174 fits on the labial of the four incisors and includes a connecting extension that involves a thick vertical post that serves to connect the three components and provide for the hinging action of gripping elements 170. In some such embodiments, the anterior part of the member 174 has a feature that provides for connection with the anterior crossbow of the MMR. In some embodiments, once gripping elements 170 have been tightened against the teeth by constricting the anterior ends of elements 170, the position is maintained either by a nut 176 that screws down to hold the right and left gripping elements tightly against the flat surface of the anterior component or by a component that holds the two anterior ends of the right and left gripping elements toward each other tightly. In some embodiments, gripping element 170 include fiducials 156 position similar to fiducials 156 of FIG. 1A.

Facial surface attach clutches allow teeth to touch during the bite and, to the extent that the teeth are exposed, permits scanning and digitization of the teeth while clutches 104 and 106 are attached. In some embodiments, facial surface attached clutches are printed with a 3D printer to expose certain teeth, or to reduce weight and size. In some situations, however, we want to reduce the effect of muscles reacting to interference. Bite trays reduce the effect of muscles reacting to interference by preventing interference between teeth.

One example embodiment of clutches 106 and 108 based on bite trays is shown in FIGS. 7C and 7D. In the example embodiment of FIGS. 7C, clutches 106 and 108 are viewed from the front. In the example embodiment, clutch 106 includes a bearing surface 160 while clutch 108 includes a bearing point 162 configured to interact with the bearing surface such that when the patient bites, their mandibular condyles are seated up and forward. Placement of the jaw position is recorded and used to establish centric bite registration while apparatus 100 is on the patient.

In the embodiment shown in FIG. 7C, bearing point 162 is guided to bearing surface 160 via bearing guide 164. In one embodiment, as is shown in FIGS. 7C, bearing guide 164 is a truncated funnel that terminates into bearing surface 160. A cross-sectional view of clutches 106 and 108 through line 164′ is shown in FIG. 7C. In one example embodiment, bearing surface 160, instead of being a flat surface, instead resembles a portion of the inside of a sphere. In other embodiments, bearing surface 160 is curved and the transition between bearing guide 164 and bearing surface 160 is more gradual. In some such embodiments, bearing guide 164 and bearing surface 160 are designed and fabricated to guide the patient's teeth into the desired bite centric position. In one embodiment, bearing guide 164 is a 3D printed component designed to foster bite centric registration for the patient under test.

In some embodiments, bite trays 106 and 108 of FIGS. 7C and 7D extend only partway back in the patient's mouth. Such an approach can be useful when the use of a full length bite tray leads to problems due to meeting in the back when the patient bites.

In some embodiments, bite trays used as clutches 106 and 108 have solid sides but the occlusal sides of the bite trays are a mesh material used to capture and contain bite registration material. In some embodiments, the bite trays are printed with a 3D printer to expose certain teeth for scanning. In some embodiments, clutches 106 and 108 are nonmetallic to foster cone beam computed tomography (CBCT) scanning of teeth while the clutches are mounted to the patient. In some such embodiments, fiducials on clutches 106 and 108 are used to provide position information on clutches 106 and 108.

In the embodiments shown in FIGS. 7E and 7F, a maxillary clutch 700 includes indexing grooves 706, such as a v-groove style. The indexing grooves 706 are included on a bottom surface 702 of the maxillary clutch 700 including a bearing surface 704. In the embodiments shown in FIGS. 7G and 7H, a mandibular clutch 710 includes indexing grooves 716, such as a v-groove style. The indexing grooves 716 are included on a top surface 712 of the mandibular clutch 710 including a bearing point 714. The indexing grooves 706 and 716, in one embodiment, are for injection of a material that allows for inter-clutch registration. In an example, the indexing grooves are approximately 2.0 mm wide at the respective surface and approximately 1.5 mm in depth.

In the embodiment shown in FIG. 7I, clutch 720 includes a plurality of adhesive bonding screws 722 to fix the clutch 720 to facial surfaces 724 of maxillary and mandibular teeth 726. In an example, bonding surface 723 attached to a screw mechanism, such as bonding screws 722, allows the bonding surface to be moved into contact with the teeth. Enamel is etched with a treatment acid and a plastic bonding adhesive is applied to the bonding screw. The bonding adhesive is then cured or set with an application of blue light. In an example, the clutch, such as a clutch illustrated in FIGS. 7A-7H and 7J-7K, is a three-dimensional (3D) customized (e.g., patient specific) framework clutch for bonding. For example, the 3D customized framework clutch is printed based on a 3D scan of a patient's teeth, so as to provide better bonding. In another example, the clutch, such as a clutch illustrated in FIGS. 7A-7H and 7J-7K, is a framework for receiving a customizable moldable and disposable insert for bonding to a patient's specific teeth.

In the embodiment shown in FIGS. 7J-K, clutch 770 includes a plurality of screws 772 to fix the clutch 770 to embrasure space between the curved proximal surface of the maxillary and mandibular teeth 726. In an example, screw mechanism, such as turn-style screws 772, allows the clutch to be positioned and contact the proximal surface of the teeth 726. In at least this example embodiment, clutch is customized to the patient's dentition. Once gripping elements 170 have been tightened against the teeth by screws 772 the gripping element 778 is positioned tightly against the flat surface of the anterior surface of the maxillary or mandibular arch. In some embodiments, gripping element 778 include fiducials 156 position similar to fiducials 156 of FIG. 1A. It should also be appreciated that the clutch 770 could be fabricated by 3-D printing using firm plastic material. Although not wanting to be bound by any particular theory clutch would take surface contours of the incisors, cuspids, bicuspids and mesial portion of the first molars. Clutch 770, once formed could be placed in the patient's mouth and adhered to tooth surfaces using compounds, such as polyvinylsiloxane (PVS).

In an example, the clutch, such as a clutch illustrated in FIGS. 7A-7H and 7J-7K, is a three-dimensional (3D) customized (e.g., patient specific) framework clutch for bonding. For example, the 3D customized framework clutch is printed based on a 3D scan of a patient's teeth, so as to provide better bonding. In another example, the clutch, such as a clutch illustrated in FIGS. 7E-7H, is a framework for receiving a customizable moldable and disposable insert for bonding to a patient's specific teeth.

In an example, a first clutch, such as shown in FIGS. 7A-7K, is affixed to the upper and a second clutch, such as shown in FIGS. 7A-7K, is affixed to the lower teeth with a firm setting polyvinyl siloxane (PVS) material (e.g., Fultar, Acu-flow, etc.). When the clutches are in place, the MMR is attached and the desired data is collected. Before removing the clutches, a firm setting PVS material is laced between the occlusal surfaces of the clutches, and the patient is closed into Centric Relation with the ‘guide ramp’ and ‘bearing point’ in contact with each other until the material sets, so as to provide the inter-clutch registration (ICR).

The clutches are removed from the patient's mouth along with the ICR and re-attached to the MMR device outside of the mouth. The ICR is repositioned between the clutches using the indexing grooves for previse placement. The MMR-clutch assembly is ready for scanning or mounting procedures with previse duplication of the actual intra- and extra-oral relationships.

In one embodiment, apparatus 100 replaces one or more fasteners 118 with linear distance monitors. A measurement computer 142 combines the scanned teeth data with the distance data from the linear distance monitors and the mandibular movement data into a bite model that helps service providers understand their patients' teeth and jaw movements. In some embodiments, one of the face bows 102 or 104 includes right and left ear canal indicators. In operation, the care provider aligns the right and left sensor ear canal indicators to ear canals. In some embodiments, the care provider aligns right and left lasers 124 to the ear canal indicators.

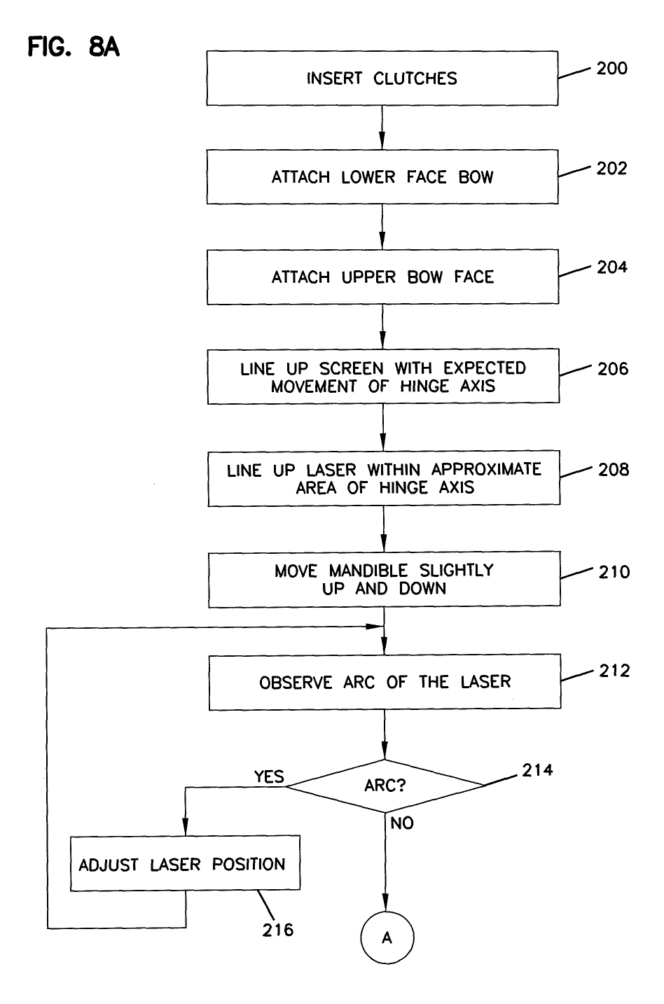

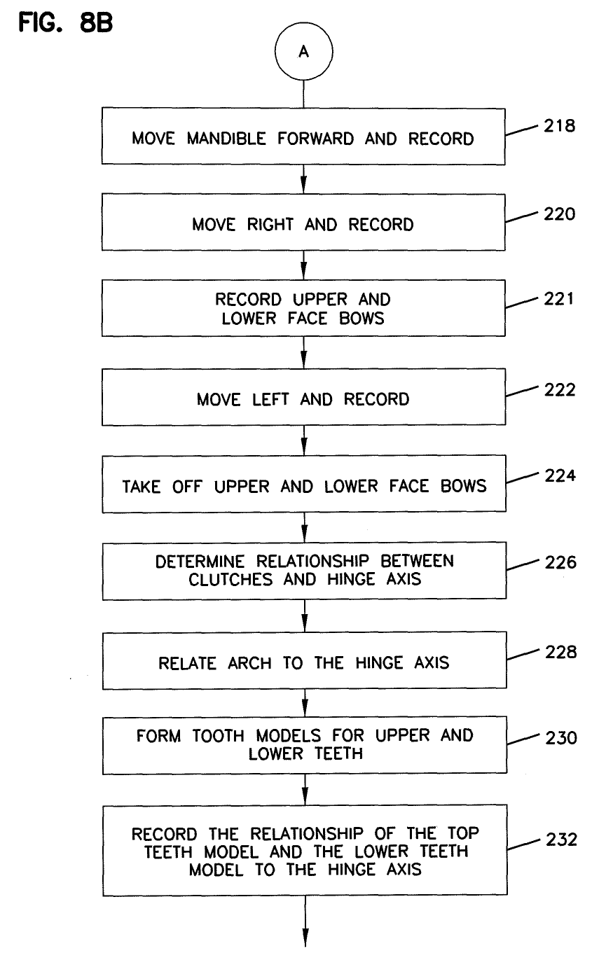

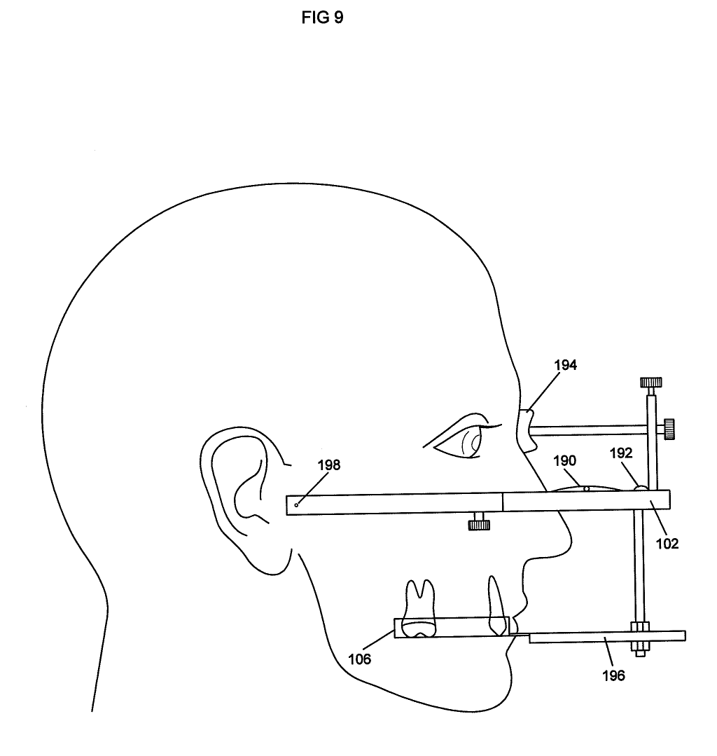

A method of relating tooth models to the condylar hinge axis is shown in FIGS. 8A and 8B. In the example shown in FIGS. 8A and 8B, at 200, the care provider inserts clutches 106 and 108 into the patient's mouth. In some embodiments, the care provider positions a patient in a chair in upright sitting position. If clutches 106 and 108 are trays, the care provider, in some embodiments, fills upper and lower clutches with a medium such as a wax. The care provider then fits the clutches to their respective teeth. In some embodiments, the care provider fits lower clutch 108 in alignment to upper clutch 106. In some embodiments, the bottoms of the trays are flat in order to deprogram jaw muscles. In other embodiments, clutches 106 and 108 are used that expose the occlusal surfaces of the teeth so interferences can be modeled in the subsequent recording.

If clutches 106 and 108 are facial surface attach clutches, the clutches are attached to the upper and lower teeth by an adhesive.

At 202, the care provider attaches lower face bow 104 to lower clutch 108 and, at 204, fastens upper face bow 102 to upper clutch 106. In some embodiments, each bow and its corresponding clutch are aligned in a horizontal plane. At 206, adjustments are made to the upper face bow 102 to position translucent screen 134 in the area of the hinge axis. At 208, adjustments are made to the lower face bow 104 to position lasers 120 and 122 in the area of the hinge axis.

At 210, the care provider moves the mandible up and down and checks, at 212, movement of the laser dots on screens 134. In some embodiments, the mandible is moved+5 mm. In other embodiments, the mandible is moved from 5 mm to 25 mm in each direction at the discretion of the care provider. If the laser dots shown on screen 134 stay in approximately the same spot, the spot marks the hinge axis. If, however, the dots move in an arc, the care provider knows at 214 that the laser dots are not on the hinge axis and, at 216, adjustments are made in the positions of lasers 120 and 124 and control moves to 212. If, at 214, the laser dots stay in approximately the same spot, each spot marks one end of the condylar hinge axis. Control moves to 218 as shown in FIG. 8B.

The centroid of each condyle changes due to translation and rotation of the mandible. This results in changes to the location of the hinge axis. At 218, the hinge axis in the starting position is now known and the care provider moves the mandible forward and records movement of the condylar axis via movement of laser light 122 and 126 on screens 134.1 and 134.2.

At 220, the care provider moves the mandible right and records movement of the condylar axis via movement of laser light 122 and 126 on screens 134.1 and 134.2. At 221, the care provider scans the upper and lower bows to record the spatial relation of the fiducials on the upper and lower bows and clutches, such prior to remove the MMR from the patient. At 222, the care provider moves the mandible left and records movement of the condylar axis via movement of laser light 122 and 126 on screens 134.1 and 134.2.

At this point, the hinge axis has been identified, and movements of the hinge axis corresponding to movements of the mandible in a variety of directions have been determined. At 224, clutches 106 and 108 are removed from the patient with upper face bow 102 and lower face bow 104 attached and the geometric relationship between the clutches and the hinge axis is determined at 226. In one example embodiment, the determining the relationship includes determining D1 and D2 as shown in FIG. 4B.

In one example embodiment, determining the relationship between the clutches and the hinge axis includes placing lower face bow 104 on a flat surface and measuring the distance between a fiducial on each side arm 116 and one of its corresponding lasers 120 and 124. In one example embodiment, determining the relationship between the clutches and the hinge axis includes placing upper face bow 102 on a flat surface and measuring the distance between fiducial 152 on each side arm 116 and anterior rod 114.

At 228, the lower arch is related to the hinge axis. In one embodiment, fiducials on upper face bow 102 and lower face bow 104 are used to determine the geometric relationship of the hinge axis to each of the fiducial locations, and from there to the teeth. In one such embodiment, fiducials 150, 152, 154 and 156 as shown in FIG. 1 are used to determine these relationships. In one embodiment, measurements are taken by via a Leap Motion Controller. In one such embodiment, measurement is performed by positioning each face bow so that the impression in the clutch is facing upward.

At 230, a tooth scan is used to form tooth models for the upper and lower teeth and, at 232, the relationship between the tooth model for the top teeth, the tooth model for the lower teeth and the hinge axis is recorded. In one such embodiment, the teeth are scanned and tooth models are superimposed on the geometry defined by fiducials 150, 152, 154 and 156. Measurements are provided in the x, y and z planes, and include measurements of pitch, yaw and roll reflective of mandible motion.

In some embodiments, the care provider instructs patient to make random movement to test system function. In some embodiments, the care provider has patients open and close their jaw several times while recording using laser 124. In some embodiments, the care provider checks to see if hinge recordings from the right and left sensors overlap. If not, in some embodiments, the care provider can optionally erase recordings and repeat hinging until the recordings overlap. In some embodiments, apparatus 100 can be calibrated by the care provider so that the hinge arcs substantially overlap (e.g., less than 5% different in amplitude along arc).

In various embodiments, the care provider checks arcs described by movement of the laser light on screens 134.1 and 134.2 on, for example, a monitor. The arcs could optionally be stored in a memory for use by another device, such as a processor in a Mandibular Movement Simulator (“MMS”) computer, as disclosed in U.S. Pat. Nos. 8,556,626 and 8,834,157. In some embodiments, the care provider queries the measurement computer 142 to show origin of arc radii with, for example, a blinking dot or target. In some embodiments, the care provider can repeat operation with different color dots to confirm duplicate result. In some embodiments, measurement computer 142 is capable of duplicating the result or calculating statistical certainty via a number of iterations.

In some embodiments, the care provider repositions horizontal laser beams, right and left, to arc radii origins, as indicated and verified by measurement computer 142. In some embodiments, the care provider repositions vertical sensors on right and left sides of apparatus 100 to align axial centers on sensors, to be coaxial with laser beams as indicated and verified on the measurement computer display and/or via a reoccurring sound that increases in frequency as the tool is moved into adjustment. In some embodiments, the care provider can choose to magnify the result on a monitor, and query the measurement computer if any errors or anomalies are indicated. If there are anomalies, in some embodiment, the care provider stores them within measurement computer 142 with a flag. If they do not, the care provider may have the patient make several protrusive movements while recording, and save those recordings.

In various embodiments, the care provider has the patient make several passes of right excursive movement, while recording. Optionally, they can check if recordings overlap. If they do not, the care provider can troubleshoot by checking equipment and adjusting or calibrating either the sensors in relation to one another or the pantograph in relation to the patent. The care provider can optionally make a note and save that note in relation to the record. Also, they can have patient make several passes of left excursive movement, while recording. The care provider may check if recordings overlap. If they do not, the care provider may troubleshoot. If they do, the care provider may note this and save the data relating to success and to the actual motion in a database in relation to the note.

As noted above, in some embodiments, the care provider removes upper face bow 102, with clutch 106 intact and sets it aside. The care provider removes lower face bow 104, with clutch 108 intact and sets it aside. They then take impressions of upper and lower arches, pour upper and lower impressions with hard stone and trim.

In some embodiments, the patient's bite is simulated digitally using feedback by comparing the bite data obtained by CBCT of the actual teeth to the modeled bite model. In some embodiments, bite data is communicated wirelessly to measurement computer 142 and storing the data therein. In some embodiments, a scan of the top teeth and the bottom teeth is stored in measurement computer 142. In some embodiments, a digital model of the patient's bite is displayed on a display of measurement computer 142. A display can include output readable by a computer. A display can include a visible display such as a video screen. In some embodiments, measurement computer 142 detects bite anomalies. Optional methods can include flagging the bite anomaly and displaying the flag. Optional methods can include controlling an automatic articulator with the measurement computer.

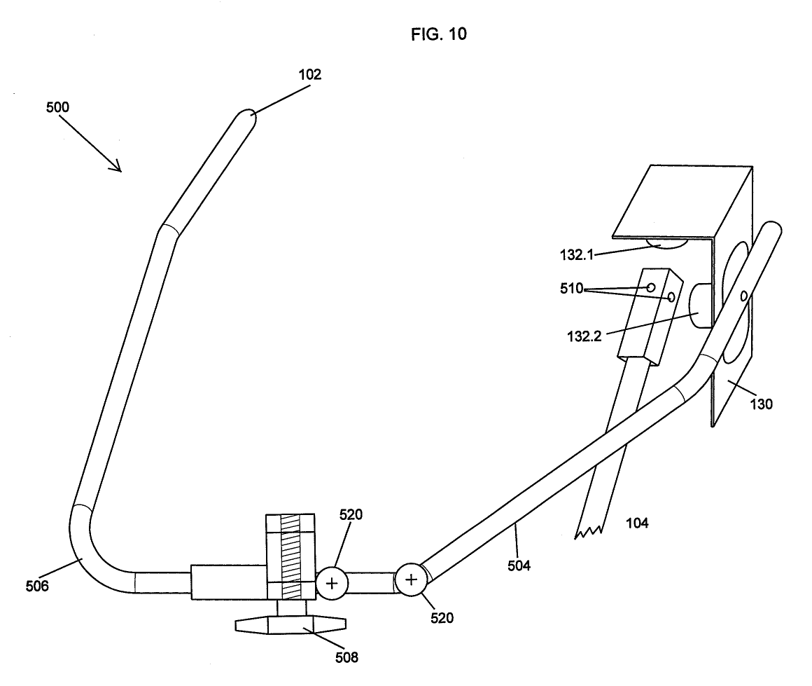

Another example embodiment of upper face bow 102 is shown in FIG. 9. In the example embodiment of FIG. 9, upper face bow 102 includes bubble levels 190 and 192. Pitch bubble level 190 detects movement off of horizontal on the transverse, or pitch, axis, while roll bubble level 192 detects movement off of horizontal on the longitudinal, or roll, axis. In the example embodiment of FIG. 9, upper face bow 102 further includes a nasion rest 194 and a clutch positioner 196. In the example shown in FIG. 9, nasion rest 194 extends from face bow 102 and, in operation, is positioned to brace upper face bow 102 against the bridge of the patient's nose when hinge axis indicator 198 is placed proximate to the hinge axis.

In operation, clutches 106 are connected to quick release clutch positioner 196 such that clutch 106 is in the correct position to take an impression of the patient's upper teeth.

FIG. 10 illustrates another embodiment of upper face bow 102 within a digital bite information collection apparatus 500. In the example embodiment of FIG. 10, upper face bow 102 includes a left side assembly 504 and a right side assembly 506. In the example shown in FIG. 10, right side assembly 506 slides into left side assembly 504 and is held in place via a fastener 508. This provides a degree of adjustability to face bow 102.

As in the example embodiment of FIG. 1, in some embodiments, a laser is used to determine how the mandibular condyle moves within the joint. In some such embodiments, one of the face bows 102, 104 includes a laser housing 110 that projects laser light 122 in a direction parallel to the sagittal plane and projects laser light 126 in a direction approximately orthogonal to the sagittal plane. In one such embodiment, such as is shown in FIG. 2, laser housing 110 includes a first laser 120 that projects light 122 upward parallel to the sagittal plane and a second laser 124 that projects light 126 out in a direction orthogonal to the sagittal plane and away from the patient's head. In one such embodiment, laser 124 is collinear. That is, laser 124 sends light in two directions: outward as in light 126 in FIG. 2 and toward the patient as in light 128 in FIG. 2. In some such embodiments, light 128 is used to trace movement of the mandibular condyle on the patient's soft tissue and can be used to guide the doctor in placing a mark in the area of the condyle. A similar laser mechanism can be used in the example embodiment shown in FIG. 10. In other example embodiments, however, lasers 120 and 124 are replaced with indicators 510 whose position in the vicinity of the hinge axis is captured by cameras 132.1 and 132.2 as shown in FIG. 10.

Still referring to FIG. 10, the face bow that does not include camera housing 130 instead includes indicators 510, such as a painted dot, reflector or an optical machine-readable representation of data such as a bar code. In the embodiment shown, camera housing 130 includes cameras 132. In some embodiments, indicators 510 are light sources such as LEDs. In one embodiment, the doctor adjusts lower face bow 104 so that passive indicators 510 are in the vicinity of the mandibular condyle. The care provider then adjusts upper face bow 102 so camera housing 130 is placed proximate indicators 510.

Although movement of the condyle is tracked using indicators 510 and cameras 132 as described above, the present subject matter is not so limited. Other optical sensors can be used to track condyle motion. The sensor fields can include, for example, position sensitive diodes (“PSDs”), but the present subject matter is not so limited.

In one embodiment, as is shown in FIG. 10, apparatus 500 includes fiducial markings 520. In the example embodiment shown in FIG. 10, fiducials 520 are used to indicate the width of upper face bow 102. In one such embodiment, fiducials 520 are scanned via, for example, a Leap Motion scanner to determine the above measurement. Such an approach is one mechanism for placing upper bow 102 and lower bow 104, and their respective clutches 106 and 108 in three-dimensional space. The Leap Motion device is available from Leap Motion, Inc., of San Francisco, Calif. In one embodiment, fiducials 520 are designed to be scanned from many different angles. In some embodiments, some or all of fiducials 520 are approximately spherical in shape and have indicia for enhancing capture of the location of the fiducial.

In one embodiment, such as is shown in FIG. 3, laser housing 110 is mounted to side arm 116 in such a way that laser housing 110 can be placed at an angle from side arm 116. In one example embodiment, laser housing 110 is mounted to side arm 116 so that laser housing 110 pivots around side arm 116. Such an approach is useful when the face bow associated with the particular side arm 116 is at an angle outside the expected range of angles. It allows the service provider to angle the laser housing to place it more in line with screens 104.

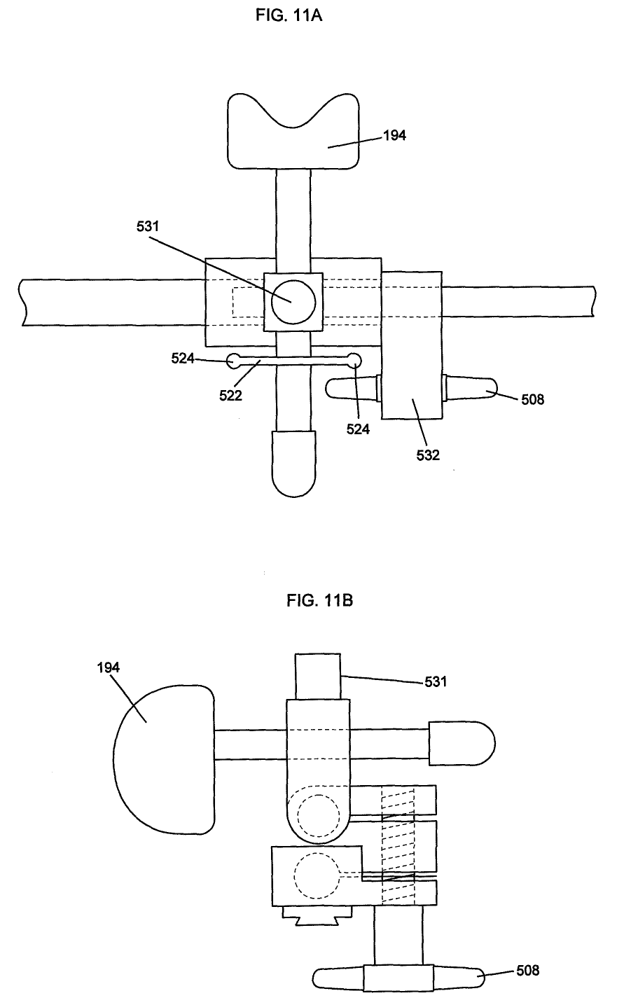

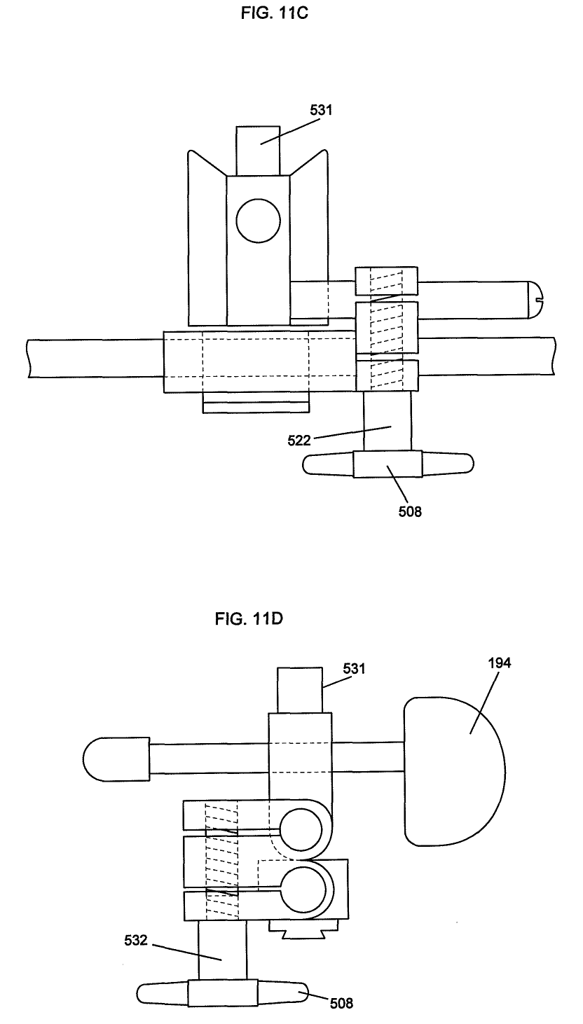

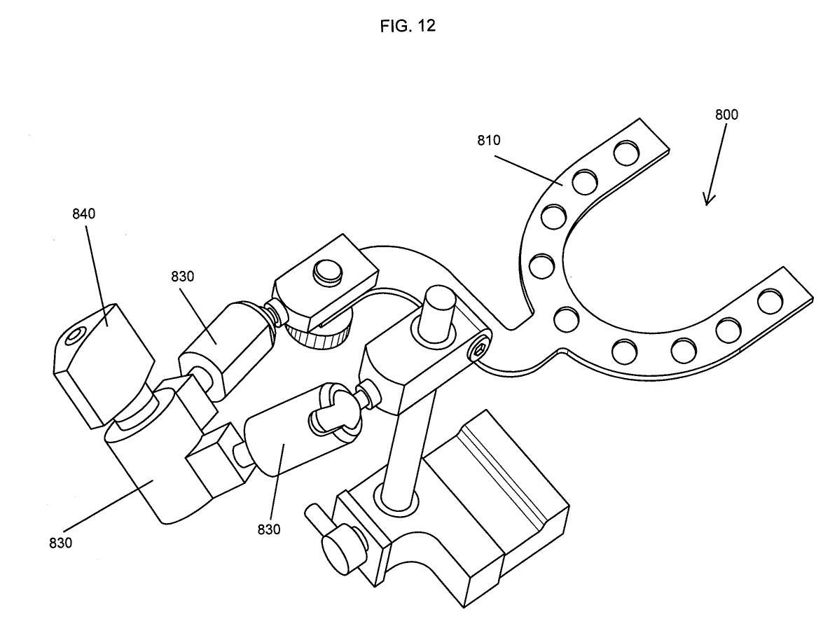

In one embodiment, clutch 106 is mounted to left side assembly 504 such that when mounted correctly, clutch 106 is in the correct position to take an impression of the patient's upper teeth. In operation, upper clutch 106 is coupled to the upper teeth and, optionally, to gingivia of a patient, such as by using a quick setting, compliant compound that can both capture the shape of the teeth and which can be released from the teeth. Lower clutch 108 is positioned by lower face bow 104 and is coupled to the bottom teeth and, optionally, to gingivia of a patient. In some embodiments, clutch 108 also uses a quick setting, compliant compound as detailed for upper clutch 106 above. One example clutch connection mechanism is shown in FIGS. 11A-11D.

In one embodiment, such as is shown in FIG. 11A, a crossbar 522 is incorporated in the clutch connection mechanism of apparatus 500. In one such embodiment, crossbar 522 is lined up with the patient's pupils during the mandibular recording process. In one embodiment, crossbar 522 is used to relate upper face bow 102 to the true horizontal. In one such embodiment, bite information collection apparatus 100 is mounted and oriented so that they are related to the true horizontal. That is, they are oriented in space the way that a person is when they stand erect. In one embodiment, this is accomplished by placing a fiducial 524 at each end of crossbar 522 and lining up fiducials 524 with the patient's pupils. One can then measure the distance between the pupils and relate the orientation of the occlusal plane of the bite, the distance between the pupils and the plane of the pupils. Such an approach allows you to determine and scale soft tissue distances.

In some embodiments, the care provider first mounts lower clutch 108 and upper clutch 106 to the patient's teeth and then attaches upper face bow 102 and lower face bow 104 to their respective clutches. In some embodiments, each clutch is connected to its face bow via clutch rods. In the embodiment shown in FIG. 1, face bows 102 and 104 include an anterior rod 114 connected to two side arms 116 and to one of the clutch rods 112. Fasteners 118 permit easy adjustment of apparatus 100.

FIG. 12 illustrates a bitefork assembly 800. In at least this example embodiment a bitefork 810 is coupled to at least one segmented arm 820 that can be positioned in an orientation desired by dental professional and each of the several segments 830 can be locked into position by a singular fastening mechanism.

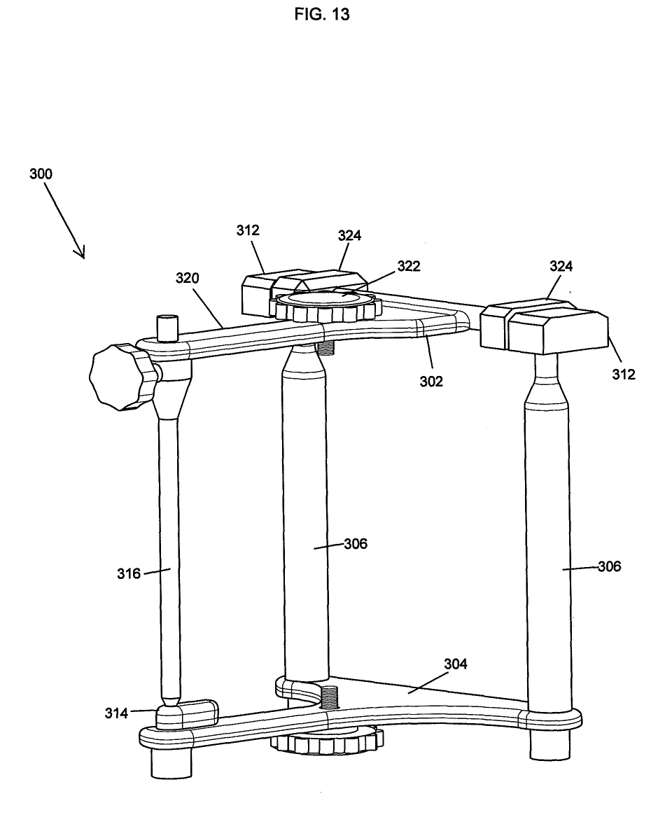

Referring now to FIG. 13 which illustrates a quick-release transfer fork assembly 184 that, in some embodiments, is connected between upper face bow 102 and clutch 106 to orient clutch 106 in a flexible manner. In some embodiments, fork assembly 184 is a single toggle connector locked in place with a single locking mechanism.

In operation, the system of FIGS. 10 and 11A-D operates as follows. Upper clutch 106 and lower clutch 108 are seated in the patient's mouth in the order of doctor preference. Mark a red dot on the patient's skin at the right and left estimated axis locations. This is just for positioning the cameras properly in the field of work. When the axis is actually located, the axis location will be marked with a black dot. Attach lower bow 104 to the lower clutch 108 and position the right and left horizontal lasers (or other position indicators) so they are in line with the red dots. Have the patient hold the upper face bow 102 on the right and left sides while the assistant moves it to position so the cameras are located properly in the field of work and the red dots are aligned with the posterior-superior aspects of the fields of work. Adjust the right side of the upper face bow 102 so the right sagittal and frontal planes are level with the eyes and/or horizon. In one example embodiment, if the eyes are level, the doctor uses the eyes for orientating the upper bow. If the eyes are not level, the doctor uses horizon. Place the nasion rest against the nose and tighten knob 531 on top of the nasion rest to secure it in the AP direction. It should be appreciated that knob 531 will set the anterior—posterior position of camera(s).

Tighten knob 532 on the anterior crossbar to secure the rotational aspect of the nasion rest and secure the lateral position of the anterior crossbar segments. Knob 532 sets the lateral-center and upward and downward position of nasion rest in addition to setting the overall width of device. In some related embodiments, knob 532 can be used to off-set camera, if applicable. Attach the “quick connect,” one turn toggle device to the insert on the upper anterior crossbar and to the insert on the upper clutch.

With all in place, tighten the “quick connect” one turn toggle to secure the anterior crossbar to the upper clutch. The total MMR placement time is estimated to be 5 minutes, leaving 10 minutes to finish the procedure and maintain an overall 15 minute total time. The process described above shows that this design will, in fact, provide for a very quick and easy placement of the MMR instrument.

The procedures for locating the axis of rotation and recording mandibular movement will not take long, so the goal of accomplishing the placement, recordings and removal in about 15 minutes appears to be very attainable. Even if the placement takes a minute or two more, the remaining tasks will allow an overall 15 minute process.

In one embodiment, when the MMR procedure has been accomplished, the MMR is removed and scanning for spatially relating the maxillary teeth to the hinge axis is accomplished as a lab procedure. With spherical fiducials on the MMR bows and the maxillary and mandibular clutches and tooth negatives, the entire instrument is scanned to capture the spatial relationship of the maxillary teeth to the hinge axis. If a given bite relationship is to be recorded, then it seems that a scan of the MMR while it is on the patient with the mandible positioned as desired would be necessary. This would be followed by scanning the mandibular clutch and tooth negatives from the tooth side. In an example, the spatial relationship are measured with calipers and recorded manually.

Dental Reconstruction using Mandible Movement Recordings

In one example embodiment, one uses the anterior stop technique to capture mandibular movement. In one such embodiment, one prepares a four thickness piece of occlusion wax that provides for the width of the maxillary anterior teeth and that provides for contact with the mandibular incisors. Generally, this piece of wax will be 1½ inches long, ½ to ¾ inches wide and ½ inches deep. Heat the wax in a water bath at 140 degrees.

In one example embodiment, prepare a two thickness piece of occlusion wax (the posterior piece) that provides for the width of the maxillary first molars at their buccal surface. Generally, this piece of wax will be 3 to 4 inches long, 1 inch wide and ⅜ inches deep. Heat the wax in a water bath at 140 degrees.

Attach apparatus 100 to the patient. After instructing the patient regarding the procedure, place the dead soft anterior piece of wax on the maxillary anterior teeth. With the thumb on the patient's chin, the index finger under the left gonial angle of the mandible and the middle finger under the right gonial angle of the mandible, manipulate the patient's mandible to cause an arcing motion of four to eight millimeters while applying only an upward pressure at the gonial angles. As this is being accomplished, one should feel the mandible seating in “rest position,” or that position of the mandible one would find it to be when the muscles of mastication are not active. The purpose of the upward pressure is only to sense if the patient moves the mandible out of rest position. The manipulation must start at rest position and the condyles must not be allowed to move out of their seated position. As this is being accomplished, one will note a smooth and freely arcing motion to the mandible. The patient is instructed to let the lower incisors strike the wax. Then the patient is instructed to “squeeze” and “bite slowly.” The care provider instructs the patient to stop biting when the maxillary and mandibular posterior teeth are about 1½ to 2 millimeters from contact. The posterior teeth cannot contact at any point or the patient will move the mandible to their habitual occlusion and the bite registration is invalid. If the procedure is satisfactory, the anterior wax piece is cooled with air from an air syringe, the piece is held in place while the patient is instructed to open, and the wax piece is place in ice water.

After the anterior wax piece has hardened in the ice water, the dead soft posterior wax piece is placed on the maxillary first molars, extending across the palate. The wax is pressed onto the occlusal surface and held in place. Then the cold, hard anterior wax piece is replaced on the maxillary anterior teeth.

Optionally, the wax is coupled to the clutches 104, 106 so that the apparatus 100 can record the relation of the teeth to the axis when the system is engaged to record. One input to record could be a foot pedal, but the present subject matter is not so limited.

During a recording period, the care provider places the fingers on the chin and the gonial angles as before and manipulates the mandible in the same manner, watching carefully to be certain the mandibular incisors fit into the registration imprints in the wax.

If the incisors fit, during another recording period, the patient is told to “squeeze” and then “bite hard evenly on both sides.” During this biting, the muscles of mastication (primarily the masseter and internal ptyergoid muscles) seat the mandibular condyles in a physiologic, seated position, which is understood to be in a superoanterior location in the mandibular fossae. This position can be recorded.

The posterior wax piece is cooled with air from the air syringe, the posterior piece is held against the maxillary molars and the patient is instructed to “open.” The anterior and posterior wax pieces are floated in water to prevent distortion. The centric relation wax bite registration (2 pieces) together with a proper transfer of the condylar axis via the apparatus 100 are used to mount the dental models in one or more dental articulators.

Centric relation is defined as the relative location of the mandible when the condyles and their properly attached articular discs are actively positioned by the closing musculature against the superoanterior areas of the posterior slopes of the articular eminences of the mandibular fossae and are also physiologically positioned transversely.

The models are “mounted in centric relation.” This is an improved mount. When combined with the improved determination of the condylar axis as set out herein, including but not limited to excursion and path data from the apparatus 100, superior and highly accurate and precise representation on an articulator can take place.

In some dental reconstruction embodiments, tracking changes in the location of teeth, changes in tooth surfaces and changes in jaw morphology are desired to plan subsequent work. In some such embodiments, a kinematic model (e.g., anatomic animation) formed by combining data from the MMR with information of the teeth captured, for instance, by scanning is used to plan dental reconstruction. In one embodiment, software executing in measurement computer 142 tracks changes in the location of teeth, changes in tooth surfaces and changes in jaw morphology when planning subsequent work.

In one example embodiment, system 140 keeps track of changes in the morphology of the jaw and in the teeth when determining the next steps to be performed. In one such embodiment, system 140 logs changes contemplated in tooth location, in the jaw and in the volume and surfaces of teeth and provides a mechanism for backing out proposed steps in the reconstruction process. This provides the dentist with a tool that can model various approaches before locking into a plan of treatment. In some such embodiments, changes in the teeth and jaw subsequent to treatment are captured and compared to the predicted changes to determine if the treatment plan is achieving the desired results.

In one embodiment, a treatment plan is modeled on system 140 and displayed to the health professional performing the dental reconstruction. In one such embodiment, a treatment plan is planned in a number of incremental steps and the incremental steps are displayed in sequence on a display. In one embodiment, each incremental step is color-coded on the display for ease of differentiation. In one such embodiment, the incremental steps can be applied to the model in sequence and backed out in the reverse of the order in which they were applied.

In one embodiment, system 140 allows you to set defaults on how much material will be removed from a tooth during treatment planning. In one embodiment, each biting surface is mapped to a grid so a doctor can know where the changes will take place. In one such embodiment, changes are recorded sequentially and teeth are numbered and divided into sections so the doctor can determine the section in which a change is made.

In one embodiment, anatomical terms are used to create a table of tooth sections and their respective teeth and to log changes in the teeth in the table. Changes to the teeth can be made to the digital model and then backed out in order to try different approaches. In another example embodiment, system 140 records the hinge axis and automatically rotates the mandible to a first bite contact each time a change is made in a tooth surface or tooth location until the maxillary and mandibular incisors couple. In one embodiment, material is removed from one or both of the teeth involved in the first bite contact according to pre-defined rules and the mandible is once again rotated to a first bite contact. In one such embodiment, material is added to teeth as well where needed.

In one embodiment, system 140 allows the doctor to change the shape of a tooth and the gum margin of the tooth, review the results and then record that change. This approach can then be used to incrementally build a comprehensive treatment plan. MMR readings can be used to model jaw function for dental jaw reconstruction as well. In one example approach, an archive of MMR scan results is maintained for each patient and is used to guide the joint reconstruction process as a function of the patient's morphology.

Custom Fossa Simulator

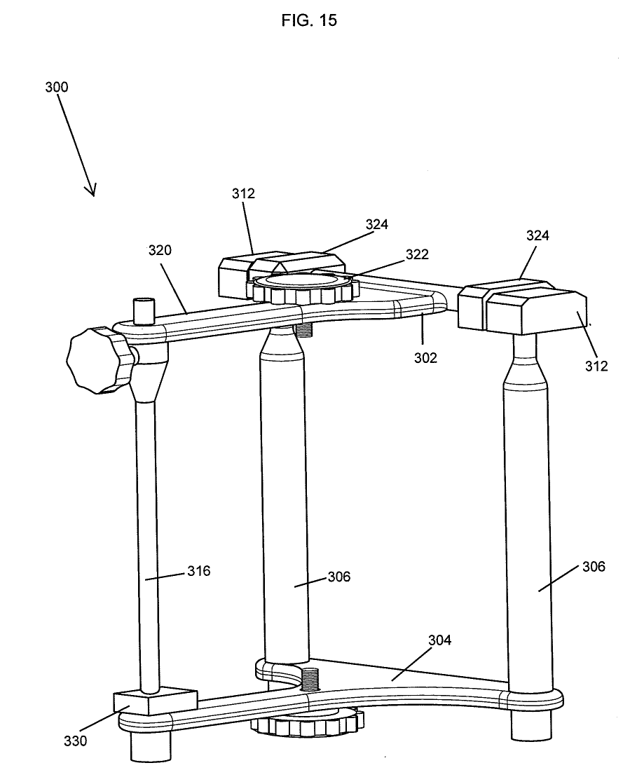

An example custom fossa simulator 300 is shown in FIGS. 13 and 15. In one embodiment, custom fossa simulator 300 is a mechanical dental articulator that uses a representation of the inferior aspects of the articular surfaces of the mandibular fossa to provide a pathway used to simulate mandibular movement. In some embodiments, the mechanical representation is a milled plastic block; in other embodiments, the mechanical representation is a 3D printed block or other such representation that can be used to guide assimilated mandibular movement in the articulator.

Custom fossa simulator 300 is configured to hold models of an upper dental arch and a lower dental arch, and to simulate movement of the jaw and interaction of the teeth in the upper and lower dental arches. Custom fossa simulator 300 includes an upper section 302 and a lower section 304. In some embodiments, the model of the upper dental arch is mounted to upper section 302 via a mounting plate while the model of the lower dental arch is mounted to lower section 304 via a second mounting plate.

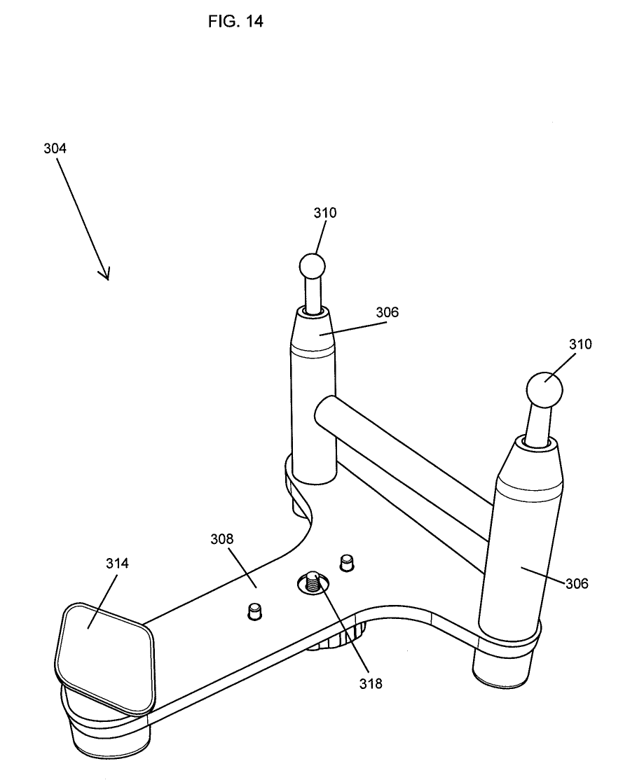

An example embodiment of the lower section 304 of simulator 300 is shown in FIG. 14. In the example shown in FIG. 14, lower section 304 includes posts 306 connected to base plate 308. Each post 306 includes a condyle 310. In the example shown in FIG. 14, each condyle 310 is approximately spherical in shape; other shapes can be used as will be discussed below. As shown in FIGS. 13 and 15, each simulator 300 includes an incisal guidance table 314 which serves to guide incisal pin 316. In one such embodiment, incisal pin 316 is adjustable as shown in FIGS. 13 and 15.

In one embodiment, the mounting plate for the lower section is attached to base plate 308 via fastener 318 while the mounting plate for the upper section is attached to upper plate 320 via fastener 322. In another related embodiment, each upper section includes two condyle guidance receptacles 324, wherein each receptacle 324 is capable of receiving a condyle guidance element 312. Receptacles 324 are located so as to situate the condyle guidance elements 312 over the condyles 310 when the condyle guidance elements are installed in the receptacles 324.

In operation, the MMR is used to locate the axis of rotation of the mandible and record mandibular movement—that is right and left lateral movements and protrusive movements so you have a record of how the jaw moves. In one embodiment, that data is then used to custom mill or 3D print a plastic block 312 that fits into receptacle 324. Surfaces of the interior of condyle guidance element 312 interact with condyle 310 to simulate operation of the condyle. In some embodiments, fossa simulator 300 provides a mechanism for moving the mandibular dental model (i.e., the physical plaster dental model) to allow the lab technician to form a crown or for the surgeon to see how the teeth fit or the orthodontist to see how the teeth fit—anything that's dealing with the treatment of dental occlusion.

In some embodiments, guidance elements 312 have shapes that, when they interact with condyles 310, mimic operation of the patient's mandibular fossae as measured by the MMR. In some such embodiments, guidance elements 312 are manufactured according to the digital data procured by the MMR recording, inserted into receptacles 324, and mounted over condyles 310. The combination of condyle 310 and guidance element 312 provides a representation of the patient's right and left mandibular fossae.

An advantage of custom fossa simulator 300 is that it can simulate mandibular movement very accurately in a mechanical procedure with a hand manipulated instrument.

In one embodiment, the condyle model used to custom mill or print block 312 is fed back into a model of the teeth to digitally simulate bite function in teeth scanned via, for instance, CBCT.

In some embodiments, condyle profiles are generated through MMR scans across a population and the profiles are used to create a library of condyle profiles characterized by physical characteristics such as the morphology of the mandibular fossae and other characteristics of the population. A best fit is then performed based on a digital model of the patient's teeth to determine the condyle profile to be used for that patient. Such an approach can be used to approximate the results of an MMR scan on the patient, but without having to perform the scan. In some embodiments, the height of each condyle 310 can be adjusted in post 306.

Another example embodiment of a fossa simulator 300 is shown in FIG. 15. In the example embodiment of FIG. 15, the incisal guidance table 314 of FIGS. 12 and 13 is replaced with a custom incisal guidance table 330. In some embodiments, custom incisal guidance table 330 is manufactured for each patient to complement condyle guidance element 312 in approximating operation of the jaw around the condyle. In operation, the MMR is used to locate the axis of rotation of the mandible and to record mandibular movement—that is right and left lateral movements and protrusive movements so you have a record of how the jaw moves. We then use that data to custom mill or 3D print an incisal guidance table 330 that attaches to an upper surface of lower section 304 as shown in FIG. 15. Surfaces of the interior of condyle guidance element 312 interact with condyle 310 while surfaces of the interior of incisal guidance table 330 interact with incisal pin 316, both of which cooperate to simulate operation of the condyle. In one embodiment condyle 310 is removable from post 306. In one such embodiment, condyle 310 is custom milled or 3D printed to operate with a standard guidance element 312 or with a custom guidance element 312.

### System and Method for Transfer to MMS With Jig

In one example embodiment, an upper face bow 102 and a lower face bow 104 are mounted to a Mandibular Movement Simulator (“MMS”) mounting jig. The lasers are aligned to targets (e.g., sensors) on the jig. In one such embodiment, the upper and lower face bows 102 and 104 are locked together. The care provider replaces the upper and lower clutches (106 and 108) into respective positions in the face bows 102 and 104 and uses the assembly to mount upper and lower trimmed stone casts to the Mandibular Movement Simulator mounting jig. At this point, the casts are mounted in a jig, and condylar axis data is also stored on the jig. This jig could be fit to a hand articulator and used to align the casts to that articulator.

In some embodiments, a care provider transfers mounted casts or molds to an MMS. This is a device that is controllable in 6 axes to simulate mandibular movement. In other words, the machine can adjust the lower mandible via servo motors. The adjustment can be along the anterior-posterior axis, around that axis, along the dorsal axis and around that axis, and along the left-right axis and around. The hinge axis is substantially parallel to the left-right axis.

In one such embodiment, upper and lower face bows 102 and 104 are attached to the jig during the transfer. In another such embodiment, upper and lower face bows 102 and 104 are used to store data relating the location of the hinge axis to the jig. Once this dimension is known, the jig can then be coupled to the MMS and the MMS can be calibrated to replicate movement according to the axis transported with the jig. This way, the care provider does not have to buy two sets of pantographs and can leave one at the measurement location and still replicate the motion at the MMS. If the care giver has one or more hand articulators, she can mount the jigs of different patients to different articulators and reconstructive work can be done for multiple patients simultaneously.

### System and Method for Transfer to MMS Without Jig

In an alternative embodiment, one can eliminate the error associated with transfer of the hinge axis location information from the measurement site, to a jig, and to the MMS, one can simply mount casts to the MMS, fit the pantographs to the MMS and the casts, and then have the MMS cycle through motions until it can replicate the arc data that's been stored in the measurement computer from the pantographs. This is achieved using feedback mechanism, such as a digital feedback system. For example, if the laser does not record a path that overlaps with the stored bite path, the simulated path is adjusted, until the simulated path replicates the stored path within a specified degree of tolerance.

### Soft Tissue Acquisition

In one embodiment, two-dimensional images captured by a camera are combined with the digital model created by combining the MMR scan with the scan of the teeth in order to build a model that includes the soft tissue of the face.

In one such embodiment, clutches 106 and 108 are attached to the patient's teeth and the patient's soft tissue is captured by taking a picture of the patient's face with a camera. The upper and lower face bows are then attached to clutches 106 and 108, respectively, an MMR scan is taken, and the relationship between the teeth, the jaw and the hinge axis is determined. In one such embodiment, determining the relationship between the teeth, the jaw and the hinge axis includes determining a bite centric relation using the MMR.

In one embodiment, a picture is taken of the patient with a panoramic camera. In another embodiment, a 3D picture is taken of the patient with clutches 106 and 108 installed.

In one embodiment, clutches 106 and 108 are attached to the patient's teeth and the patient's soft tissue is captured by taking a picture of the patient's face with a camera. The position of the clutches and the patient's teeth are then determined using, for example, cone beam X-Rays or CBCT with the teeth in a centric relation. In one such embodiment, the hinge axis is then determined based on the centric relation. The data on the underlying bone structure determined from the cone beam X-Rays or the CBCT process is then combined with the data from the images captured by the camera to build a model including soft tissue.

In one embodiment, real-time 3D imaging is used to capture movement of the soft tissue in real-time around the teeth and facial bone structure. One such real-time imaging technique is described by Karpinsky et al. in “High-resolution, real-time 3D imaging with fringe analysis,” published Mar. 1, 2012 in the Journal of Real-Time Image Processing, Volume 7, Issue 1 , pp 55-66, the description of which is incorporated herein by reference. One advantage of such a camera is the ability to capture anatomical features in real-time.

In one real-time 3D imaging embodiment, fiducials such as light emitting diodes (LED) or other indicia are placed on clutches and the clutches are seated in the patient's mouth. The indicia are designed to be easily detected by the real-time 3D imaging camera, serving to capture the position of the clutches in the images.

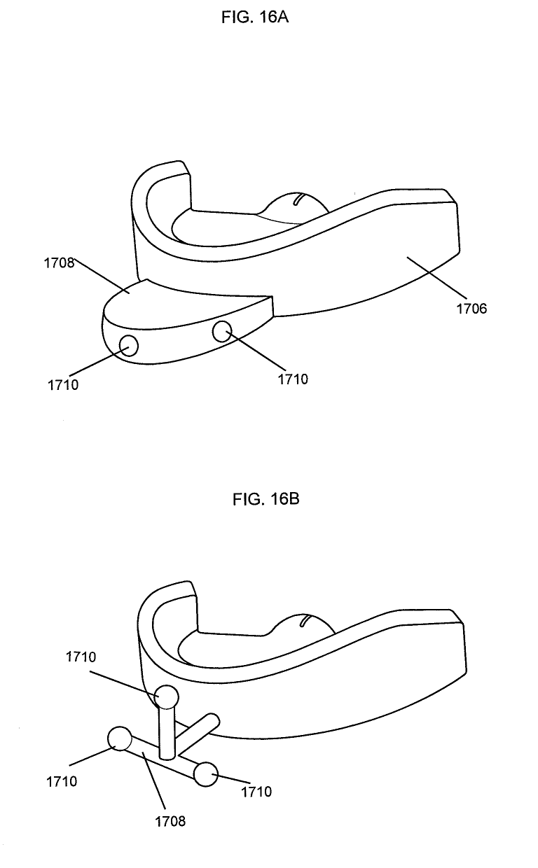

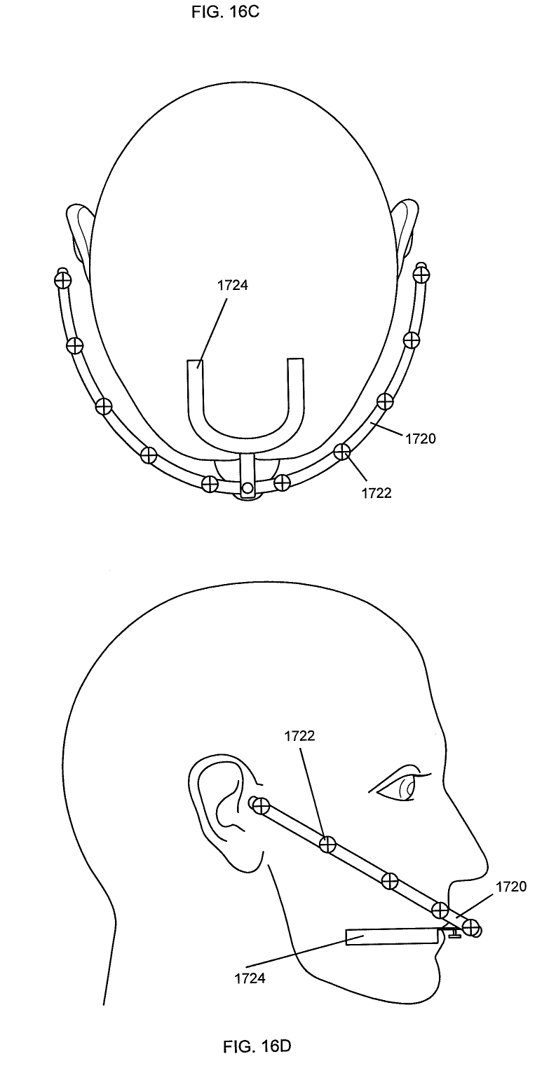

One embodiment of a clutch that can be used in a real-time 3D imaging embodiment is shown in FIG. 16A. In the example embodiment of FIG. 16A, clutch 1706 is a bite-tray style clutch used for the upper teeth. Clutch 1706 includes an anterior fiducial unit 1708 that extends from the body of clutch 1706 through the patient's mouth so that it can be seen in the captured real-time 3D image. Each anterior fiducial unit 1708 includes fiducials 1710 disposed thereon and used to determine the location and orientation of clutch 1706 in the patient's mouth.

Another example embodiment of a clutch that can be used in a real-time 3D imaging embodiment is shown in FIG. 16B. Once again, in the example embodiment of FIG. 16B, clutch 1706 is bite-tray style clutch used for the upper teeth. Clutch 1706 includes an anterior fiducial unit 1708 that extends from the body of clutch 1706 through the patient's mouth so that it can be seen in the captured real-time 3D image. Each anterior fiducial unit 1708 includes fiducials 1710 disposed thereon and used to determine the location and orientation of clutch 1706 in the patient's mouth. In the example shown in FIG. 16B, the fiducials 1710 are located in more than one plane. Lower clutches with similar designs are used to capture movement of the lower teeth and jaw. In some embodiments, clutches that connect to or are bonded to facial surfaces of the teeth are used effectively as well in a real-time 3D imaging embodiment.

In at least one embodiment, upper and lower clutches similar to those discussed above are seated in the patient's mouth and a real-time 3D imaging system is used to capture anatomical features of the face and the location and orientation of the clutches, and, via the clutches, the patient's teeth. The jaw is placed in a centric relation and a screw axis is extrapolated based on the centric relation. In some embodiments, the doctor manipulates the lower jaw in the manner described previously to capture movement of the jaw with a properly seated condyle. Cone-beam computed tomography is then used to capture the underlying bone structure.

In another example real-time 3D imaging embodiment, as is shown in FIGS. 16C and 16D, fiducials 1722 extend along a bow 1720 attached to a clutch 1724 mounted on the patient's lower teeth. In one such embodiment, bow 1720 is oriented toward where the doctor perceives to be the hinge axis. Once again, a real-time 3D imaging system is used to capture anatomical features of the face and the location and orientation of the clutch 1724, and, via clutch 1724, the patient's lower teeth. The jaw is placed in a centric relation and a screw axis is extrapolated based on the centric relation. In some embodiments, the doctor manipulates the lower jaw in the manner described previously to capture movement of the jaw with a properly seated condyle. Cone-beam computed tomography is then used to capture the underlying bone structure.

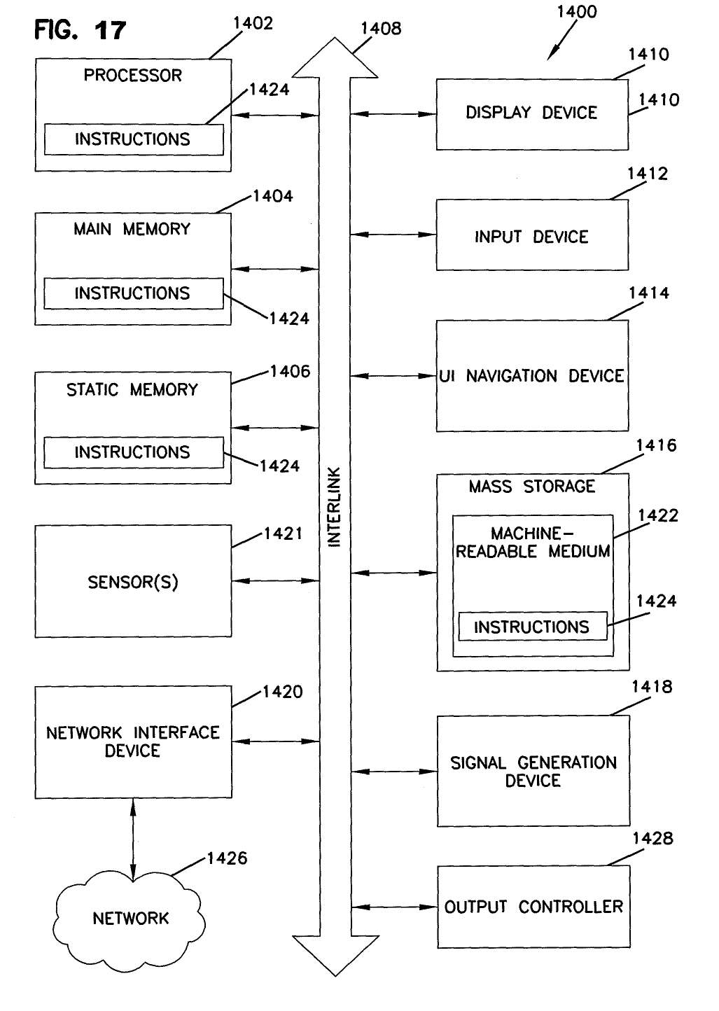

FIG. 17 illustrates a block diagram of an example circuit 1400 which can be used in conjunction with any of the examples discussed herein. The circuit 1400 can operate as a standalone device or can be connected (e.g., networked) to other circuits. The circuit 1400 can form all or a part of a personal computer (PC), a tablet PC, a set-top box (STB), a Personal Digital Assistant (PDA), a mobile telephone, a web appliance, a network router, switch or bridge, or any machine capable of executing instructions (sequential or otherwise) that specify actions to be taken by that machine. The term “circuit” can include any collection of circuits that individually or jointly execute a set (or multiple sets) of instructions to perform any one or more of the systemologies discussed herein, such as cloud computing, software as a service (SaaS), other computer cluster configurations. Insofar as circuit embodiments include software, such software can reside on a machine readable medium. Software, when executed by hardware, can cause the hardware to perform a function.

Circuit (e.g., computer system) 1400 can include a hardware processor 1402 (e.g., a central processing unit (CPU), a graphics processing unit (GPU), a hardware processor core, or any combination thereof), a main memory 1404 and a static memory 1406, some or all of which can communicate with each other via an interlink (e.g., bus) 1408. The circuit 1400 can further include a display unit 1410, an alphanumeric input device 1412 (e.g., a keyboard), and a user interface (UI) navigation device 1414 (e.g., a mouse). The display unit 1410, input device 1412 and UI navigation device 1414 can be a touch screen display. The circuit 1400 can additionally include a storage device 1416 (e.g., a drive unit), a signal generation device 1418 (e.g., a speaker), a network interface device 1420, and one or more sensors 1421, such as a global positioning system (GPS) sensor, compass, accelerometer, or other sensor. The circuit 1400 can include an output controller 1428, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1416 can include a machine readable medium 1422 on which is stored one or more sets of data structures or instructions 1424 (e.g., software) embodying or utilized by any one or more of the techniques or functions described herein. The instructions 1424 can also reside, completely or at least partially, within the main memory 1404, within static memory 1406, or within the hardware processor 1402 during execution thereof by the circuit 1400. In an embodiment, one or any combination of the hardware processor 1402, the main memory 1404, the static memory 1406, or the storage device 1416 can constitute machine readable media.

While the machine readable medium 1422 is illustrated as a single medium, the term “machine readable medium” can include a single medium or multiple media (e.g., a centralized or distributed database, and/or associated caches and servers) that are configured to store the one or more instructions 1424.

The term “machine readable medium” can include any medium that is capable of storing, encoding, or carrying instructions for execution by the circuit 1400 and that cause the circuit 1400 to perform any one or more of the techniques of the present disclosure, or that is capable of storing, encoding, or carrying data structures used by or associated with such instructions. Non-limiting machine readable medium embodiments can include solid-state memories, and optical and magnetic media. In an embodiment, a massed machine readable medium comprises a machine readable medium with a plurality of particles having resting mass. Specific embodiments of massed machine readable media can include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks. Machine readable medium do not include carrier waves or other propagated data signals.

The instructions 1424 can further be transmitted or received over a communications network 1426 using a transmission medium via the network interface device 1420 utilizing any one of a number of transfer protocols (e.g., frame relay, internet protocol (IP), transmission control protocol (TCP), user datagram protocol (UDP), hypertext transfer protocol (HTTP), etc.). Embodiments of communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), peer-to-peer (P2P) networks, among others. The network interface device 1420 can include one or more physical jacks (e.g., Ethernet, coaxial, or phone jacks) or one or more antennas to connect to the communications network 1426. The network interface device 1420 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the circuit 1400, and includes digital or analog communications signals or other intangible medium to facilitate communication of such software. The example circuit 1400 can include a digital bite replicator. The example circuit 1400 can include a computer such as to store a measured distance. Sensors 1421 can include sensors such as sensors 132.1, 132.2, 132.3, and 132.4 in FIG. 1. It should be appreciated that other sensors that can capture images or capture variations in surface topography could be employed in this portion of the bite information collection apparatus.

Examples herein provide a digital dental diagnostic system. The above detailed description includes references to the accompanying drawings, which form a part of the detailed description. The drawings show, by way of illustration, specific embodiments in that may be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects or a priority of importance between similar parts.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature and gist of the technical disclosure. The Abstract is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim.

1. A bite information collection apparatus comprising:
an upper face bow connected to an upper clutch and
a lower face bow connected to a lower clutch,
wherein the upper clutch comprises at least one fiducial and is configured to releasably connect to the upper teeth of a patient and wherein the lower clutch comprises at least one fiducial and is configured to releasably connect to the bottom teeth of the patient and wherein the upper face bow and lower face bow each comprise at least one fastener for adjusting the bite information collection apparatus.

2. The bite information collection apparatus of claim 1, wherein the upper face bow comprises an anterior rod connecting two opposing side arms.

3. The bite information collection apparatus of claim 1, wherein the lower face bow comprises an anterior rod connecting two opposing side arms.

4. The bite information collection apparatus of claim 1, wherein the upper clutch comprises a compound for reproducing the shape of the upper teeth.

5. The bite information collection apparatus claim 1, wherein the lower clutch comprises a compound for reproducing the shape of the lower teeth.

6. The bite information collection apparatus of claim 1, wherein the upper clutch is connected to the upper face bow by a clutch rod.

7. The bite information collection apparatus of claim 1, wherein the lower clutch is connected to the lower face bow by a clutch rod.

8. The bite information collection apparatus of claim 1, wherein the upper face bow comprises a laser housing.

9. The bite information collection apparatus of claim 1, wherein the lower face bow comprises a laser housing.

10. The bite information collection apparatus of claim 1, wherein the upper face bow comprises at least one fastener.

11. The bite information collection apparatus of claim 1, wherein the lower face bow comprises at least one fastener.

12. The bite information collection apparatus of claim 1, wherein the upper face bow comprises at least one fastener to secure the horizontal plane of the upper face bow.

13. The bite information collection apparatus of claim 1, wherein the lower face bow comprises at least one fastener to secure the horizontal plane of the lower face bow.

14. The bite information collection apparatus of claim 1, wherein the upper clutch comprises at least two fiducials.

15. The bite information collection apparatus of claim 1, wherein the lower clutch comprises at least two fiducials.

16. The bite information collection apparatus of claim 1, wherein the upper face bow comprises a camera housing.

17. The bite information collection apparatus of claim 1, wherein the lower face bow comprises a camera housing.

18. The bite information collection apparatus of claim 1, wherein the upper face bow further comprises a translucent screen.

19. The bite information collection apparatus of claim 1, wherein the lower face bow further comprises a translucent screen.

20. The bite information collection apparatus of claim 1, wherein the upper clutch is connected to the upper face bow by an upper magnetized component.

21. The bite information collection apparatus of claim 1, wherein the lower clutch is connected to the lower face bow by a lower magnetized component.

22. The bite information collection apparatus of claim 1, wherein the upper magnetized component comprises a first metallic portion and a second metallic portion.

23. The bite information collection apparatus of claim 1, wherein the lower magnetized component comprises a first metallic portion and a second metallic portion.

24. The bite information collection apparatus of claim 1, wherein the upper face bow further comprises a translucent screen.

25. The bite information collection apparatus of claim 1, wherein the lower face bow further comprises a translucent screen.

26. The bite information collection apparatus of claim 1, further comprising a measurement computer.

27. The bite information collection apparatus of claim 1, wherein the upper clutch is releasbly connected to the upper teeth via an adhesive compound.

28. The bite information collection apparatus of claim 1, wherein the lower clutch is releasbly connected to the lower teeth via an adhesive compound.

29.-42. (canceled)

43. A method of collecting bite information, the method comprising:
activating laser lights on a bite information collection apparatus;
capturing a plurality of images using a plurality of optical sensors of the bite information collection apparatus while the patient's mandible is moved about the hinge axis, the plurality of images including translucent screens of the bite information collection apparatus;
analyzing, by a computing device, the plurality of images to determine whether the bite information collection apparatus is aligned with the hinge axis; and
when determined that the bite information collection apparatus is not aligned with the hinge axis, displaying a user interface including instructions for adjusting the bite information collection apparatus to align with the hinge axis.

44.-56. (canceled)
